(19) United States
(12) Patent Application Publication
(10) Pub. No.: US 2018/0250321 A1
(43) Pub. Date: Sep. 6, 2018

(54) COMPOSITIONS AND METHODS FOR RESTORING THE IMMUNE SYSTEM

(71) Applicant: ORPHEUS THERAPEUTICS, INC., San Francisco, CA (US)

(72) Inventor: MARTIN SCHROEDER, San Francisco, CA (US)

(21) Appl. No.: 15/904,155

(22) Filed: Feb. 23, 2018

Related U.S. Application Data
(60) Provisional application No. 62/464,272, filed on Feb. 27, 2017.

(51) Int. Cl. A61K 31/7028; A61K 47/68; A61K 45/06; A61P 35/04

(52) U.S. Cl. CPC A61K 31/7028; A61K 47/6807; A61K 2039/505; A61P 35/04; A61K 45/06

(57) ABSTRACT

A pharmaceutical composition for shifting a Th2 immune response to a Th1 response by natural killer T cells (NKT cells) including a therapeutically effective amount of a glycolipid of one or more of Formulas II-V or a pharmaceutically acceptable salt thereof, and a pharmaceutical carrier is provided.

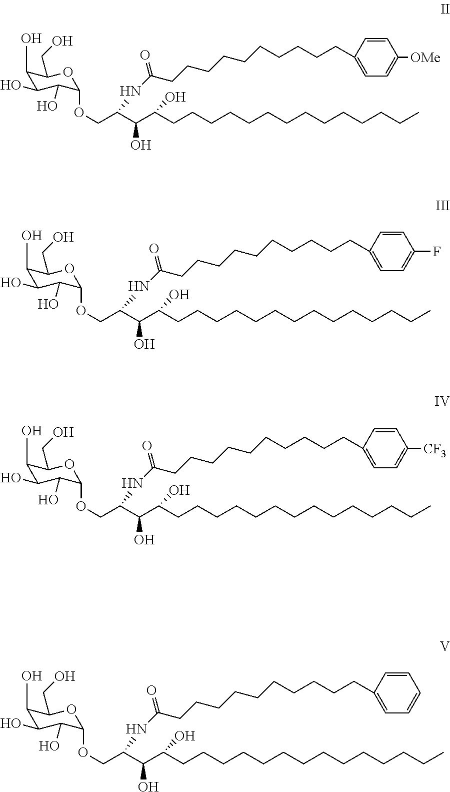 [SMILES: CCCCCCCCCCCCCC[C@@H](O)[C@@H](O)[C@H](COC1OC(CO)C(O)C(O)C1O)NC(=O)CCCCCCCCCCc1ccc(OC)cc1 ; CCCCCCCCCCCCCC[C@@H](O)[C@@H](O)[C@H](COC1OC(CO)C(O)C(O)C1O)NC(=O)CCCCCCCCCCc1ccc(F)cc1 ; CCCCCCCCCCCCCC[C@@H](O)[C@@H](O)[C@H](COC1OC(CO)C(O)C(O)C1O)NC(=O)CCCCCCCCCCc1ccc(C(F)(F)F)cc1 ; CCCCCCCCCCCCCC[C@@H](O)[C@@H](O)[C@H](COC1OC(CO)C(O)C(O)C1O)NC(=O)CCCCCCCCCCc1ccccc1]

### CROSS-REFERENCE TO RELATED APPLICATION

This application claims the benefit under 35 U.S.C. § 119 (e) of U.S. Provisional Application No. 62/464,272, filed on Feb. 27, 2017 in the United States Patent and Trademark Office, and all the benefits accruing therefrom, the content of which is incorporated herein in its entirety by reference.

### FIELD OF THE INVENTION

The present invention relates generally to small molecule compounds and more specifically to the use of glycolipid compounds for restoring an immune response.

### BACKGROUND OF THE INVENTION

The immune system and cancer interact in a dynamic process, consisting of three phases, sometimes referred to as the 3 E's (1):

Elimination (immune system eradicates cancer cells)

Equilibrium (immune system controls cancer outgrowth)

Escape (cancer overwhelms the immune system).

Cancer immunoediting is an extrinsic tumor suppressor mechanism that commences after cellular transformation has occurred and intrinsic tumor suppressor mechanisms have failed (FIG. 1). Cancer immunoediting consists of three sequential phases: elimination, equilibrium, and escape. In the elimination phase, innate and adaptive immunity destroy developing tumors before they are clinically apparent. If this phase goes to completion, then the host remains cancer free. If, however, a cancer cell is not destroyed, the cell may then enter the equilibrium phase, in which further growth is prevented by immunologic mechanisms. T cells, IL-12, and IFN-γ are required to maintain tumor cells in a state of dormancy, whereas Natural Killer (NK) cells and molecules that participate in the recognition or effector function of cells of immunity are not required. This suggests that equilibrium is a function of adaptive immunity only. Editing of tumor immunogenicity occurs in the equilibrium phase. Equilibrium may also represent an end stage of the cancer immunoediting process, and may prevent growth of cancers for the lifetime of the host. However, as a consequence of constant immune selection pressure placed on genetically unstable tumor cells held in equilibrium, tumor cell variants may emerge that (i) are no longer recognized by adaptive immunity (antigen loss variants or tumors cells that develop defects in antigen processing or presentation), (ii) become insensitive to immune effector mechanisms, or (iii) induce an immunosuppressive state within the tumor microenvironment. These tumor cells may then enter the escape phase, in which their growth is no longer prevented by immunity (2, 3).

Immunotherapies while effective in many respects when used as a monotherapy still have many shortcomings. For example, immunotherapies are still not able to produce long-lasting durable clinical responses in many cancer patients. Human tumors are very heterogeneous and develop clinical resistance to monotherapies. Results from preclinical studies have demonstrated that efficient antitumor strategies must focus on hitting different therapeutic targets concurrently.

Thus, there is a need for combination therapies by activating immune cell partners to enhance durability of the initial clinical response.

### SUMMARY OF THE INVENTION

The present invention is based on the seminal discovery that certain glycolipids are effective in displacing Th2 biasing tumor-specific glycolipids (see e.g., FIG. 12) from CD1d and stimulating natural killer T cells (NKT cells) to shift to a Th1 immune response profile. Displacing tumor glycolipids provided by the invention, when bound to CD1d, can redirect NKT cell response to a Th1 polarized response facilitating cancer immune surveillance and tumor cell death.

In one embodiment, the invention provides a pharmaceutical composition that includes a therapeutically effective amount of a glycolipid of Formula I, II, III, IV, or V, or a pharmaceutically acceptable salt thereof. A therapeutically effective amount of glycolipid is the amount effective in stimulating a shift of from a Th2 immune response to a Th1 immune response, the glycolipid has a human CD1d EC50 binding affinity of about 5× less than the corresponding tumor-specific glycolipid EC50 binding affinity to human CD1d, and results an increase in interferon-gamma production of at least about 2-fold within about 20 hours of administration, as compared with levels of interferon-gamma production prior to administration.

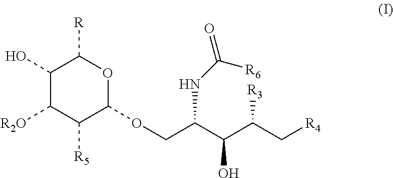

R is COOR₁ or CH₂OR₁.

R₁ is H or an alkyl group.

R₂ is H or SO₃⁻.

R₃ is H or OH.

R₄ is H, an alkyl group, an alkenyl group or an oxaalkyl group.

R₅ is OH, acetamido or a halogen atom.

R₆ is X-A.

A is dialkyl phenyl;

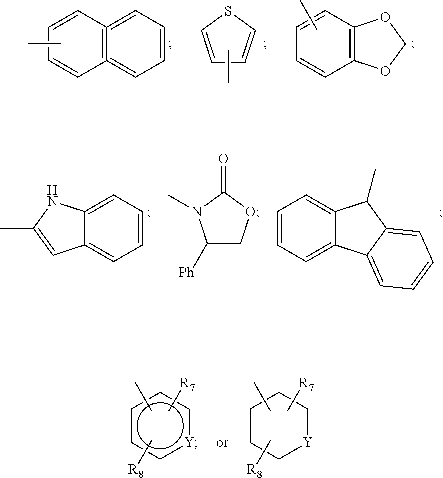

X is alkyl, alkenyl, alkoxy, thioalkoxy, substituted furan, or unsubstituted furan.

Y is N or C.

R₇ is halogen, H, phenyl, alkyl, alkoxy, nitro or CF₃.

R₈ is methyl or H.

In another embodiment, the invention provides a pharmaceutical composition that includes a therapeutically effective amount of a glycolipid of one or more of the following formulas: Formula II, III, IV, and V or a pharmaceutically acceptable salt thereof.

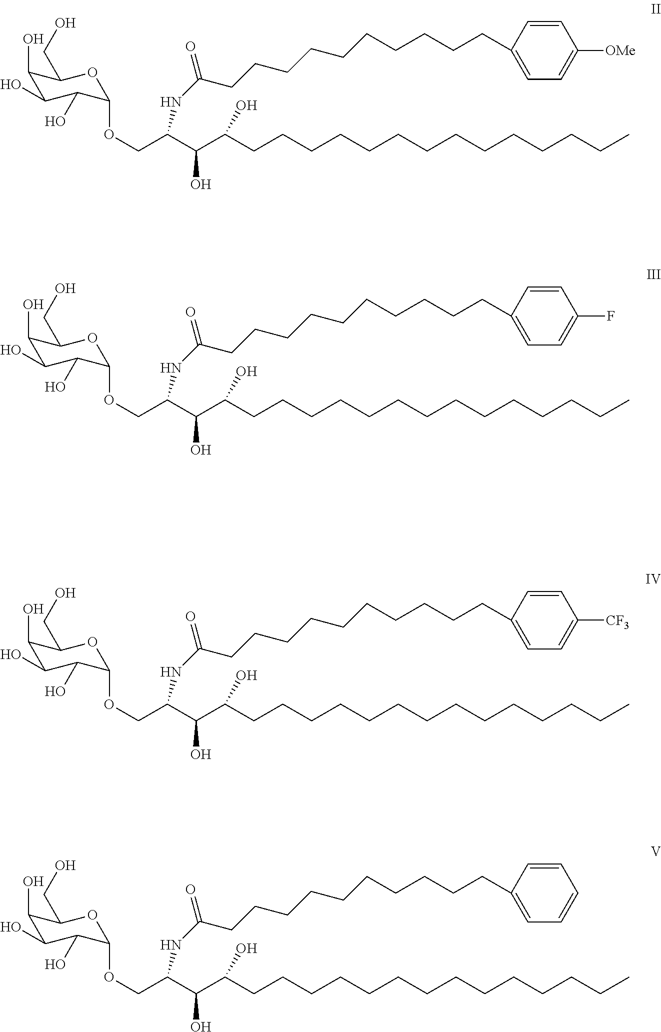 [SMILES: CCCCCCCCCCCCCC[C@@H](O)[C@@H](O)[C@H](COC1OC(CO)C(O)C(O)C1O)NC(=O)CCCCCCCCCCc1ccc(OC)cc1 ; CCCCCCCCCCCCCC[C@@H](O)[C@@H](O)[C@H](COC1OC(CO)C(O)C(O)C1O)NC(=O)CCCCCCCCCCc1ccc(F)cc1 ; CCCCCCCCCCCCCC[C@@H](O)[C@@H](O)[C@H](COC1OC(CO)C(O)C(O)C1O)NC(=O)CCCCCCCCCCc1ccc(C(F)(F)F)cc1 ; CCCCCCCCCCCCCC[C@@H](O)[C@@H](O)[C@H](COC1OC(CO)C(O)C(O)C1O)NC(=O)CCCCCCCCCCc1ccccc1]

In another embodiment, the invention provides a pharmaceutical composition that includes a therapeutically effective amount of a glycolipid of one or more of the following formulas: Formula I, II, III, IV, and V or a pharmaceutically acceptable salt thereof.

In another embodiment, the pharmaceutical composition further includes a therapeutic antibody, an antibody-drug conjugate, a radioimmunotherapy agent, a small molecule therapeutic agent, or an immune stimulating agent, such as a Toll-Like Receptor (TLR) agonist.

In another embodiment, the pharmaceutical composition is administered to a subject having cancer.

In another embodiment, the cancer is a tumor, a cancerous lesion, cancerous lymph node, or a circulating tumor cell (CTC).

In another embodiment, the cancer is selected from the group consisting of hematopoietic malignancies, prostate cancer, renal cancer, breast cancer, ovarian cancer, multiple myeloma, melanoma, colon cancer, lung cancer, cervical cancer, and certain neurological tumors, or neurological tumors, and metastases thereof.

In another embodiment, the therapeutic antibody further includes a drug to form an antibody-drug conjugate.

In another embodiment, the pharmaceutical composition includes a therapeutic antibody, and the glycolipid is conjugated to the therapeutic antibody to form an antibody-glycolipid conjugate.

In another embodiment, the invention provides a method of treatment that includes administering one of the foregoing pharmaceutical compositions to a subject in need thereof.

In another embodiment, the subject has cancer.

In another embodiment, the cancer is selected from the group consisting of hematopoietic malignancies, prostate cancer, renal cancer, breast cancer, ovarian cancer, multiple myeloma, melanoma, colon cancer, lung cancer, cervical cancer, and certain neurological tumors, and metastases thereof.

In another embodiment, the glycolipid is administered intravenously or by subcutaneous injection.

In another embodiment, the glycolipid and therapeutic antibody are administered simultaneously or separately.

In another embodiment, the administration is by the same or by different routes of administration.

In another embodiment, the glycolipid and therapeutic antibody are combined in a single formulation.

In another embodiment, the glycolipid is administered prior to, simultaneously with or following administration of the therapeutic antibody.

Other aspects and advantages of the invention will be apparent from the following description and the appended claims.

### BRIEF DESCRIPTION OF THE DRAWINGS

In the following figures, OT-0011 is equivalent to Formula II, OT-0010 is equivalent to Formula III, and OT-0012 is equivalent to Formula IV.

FIG. 1. Cancer immunoediting. Outline of how cancer evades immune surveillance.

FIG. 2. Combination of immunology-oncology approach. Targeting immune system checkpoints.

FIG. 3. Restoring immune protection.

FIGS. 4A-4D. Key nodes of the immune system to target with a combination immune-oncology therapeutics.

FIG. 5A-5C. Antibody killing mechanisms.

FIG. 6. Targeting natural killer T-cells (NKT cells) to relieve immune system checkpoint blockade.

FIG. 7. NKT cells kill tumor cells expressing CD1d or kill tumor cells by interacting with immune cell partners.

FIG. 8. CD1d expression in human tissue.

FIG. 9. NKT cells interact with immature dendritic cells in the presence of glycolipids.

FIG. 10. Current immune checkpoint therapeutic strategy.

FIG. 11. Tumors shed glycolipids resulting in immune system escape.

FIG. 12. Glycolipid gangliosides expressed by human cancer cells.

FIG. 13. Immunotherapeutic strategies targeting tumor-associated glycolipid gangliosides.

FIG. 14. Glycolipid recognition by NKT cells.

FIG. 15. NKT cells recognize glycolipids presented by CD1d. A common structural motif: a hydrophilic glycan head group and two hydrophobic acyl chains.

FIG. 16. GD3 ganglioside immune system blockade.

FIGS. 17A-17B. NKT cell response to OT-0010 glycolipid presented by HeLa-CD1d cancer cell.

FIG. 18. OT-0010 reverses GD3 ganglioside immune blockade by shifting NKT cells to a Th1 response.

FIGS. 19A-19B. OT-0010 combination immuno-oncology therapeutic tumor challenge mouse model.

FIGS. 20A-20C. OT-0010 human NKT cell TCR binding.

FIGS. 21A-21C. OT-0010 human IFN-γ production.

FIG. 22. Tumor-associated antigens targeted by therapeutic antibodies.

FIG. 23. Clear renal cell carcinoma over expresses CD1d in a disease dependent manner.

FIG. 24. NKT cells interact directly with prostate cancer cells via CD1d.

FIGS. 25A-25C. GD3 inhibits α-GalCer-induced cytokine production in vivo. (A), wild-type (WT) C57BL/6 mice were treated intravenously with 1 μg of either α-GalCer in the presence or absence of the indicated concentrations of GD3, and serum samples were obtained at the indicated time points after injection for ELISA analyses of IFN-γ, IL-4 (B), and IL-12 (C) concentrations. These data are expressed as the mean±SD of 2 different dilutions of pooled sera. In all figures, these data represent 1 of 2 or more experiments with similar results (23).

FIG. 26. CD1d captures OT-0010 faster than α-GalCer, which shows the enhanced avidity of OT-0010 compared α-GalCer.

FIG. 27. OT-0010 dose escalation study with OV2944-HM-1 ovarian cancer cell (HM-1/luc+/CD1d+/PD-L1+) in WT C57BL/6 mice. After tumor development mice were administered by intravenous injection at 0.1 μgm glycolipid/mouse.

FIGS. 28A-28D. OT-0010 improves overall survival in lung (TC1), breast (4T1) and melanoma (B16) cancer models. Three days after tumor initiation, mice were intravenously injected with indicated glycolipids at 0.1 μgm glycolipid/mouse or vehicle once a week for 4 weeks.

FIGS. 29A-29C. OT-0010 induces potent adaptive immune response and immune memory effect.

FIGS. 30A-30C. OT-0010 promotes potent NKT cell innate (direct) tumor cell killing.

FIGS. 31A-31D. OT-0010 improves overall survival in colon cancer (CT26) model. P8032 in this figure refers to Salmonella/human surviving TAA (PsifB::sseJ-coSVN) construct.

### DETAILED DESCRIPTION OF THE INVENTION

The Natural killer T cell (NKT cell) is one of the key players that link the innate and adaptive immune systems. NKT cells are unconventional innate-like T lymphocytes that possess the ability to quickly respond to antigenic stimulation, and rapidly produce copious amounts of cytokines and chemokines. This rapid effect response can modulate the quality and quantity of both downstream innate and adaptive immunity, and is important in influencing host immune responses to cancer. NKT cells are a heterogeneous subset of specialized T cells. These immune cells exhibit an innate cell-like feature of quick response to antigenic exposure in combination with an adaptive cell's precision of antigenic recognition and diverse effector responses. Like conventional T cells, NKT cells undergo thymic development and selection, and possess a T cell receptor (TCR) to recognize antigens. Unlike conventional T cells however, the TCR expressed by NKT cells recognize glycolipid antigens presented by the conserved and non-polymorphic MHC class I1-like transmembrane glycoprotein CD1d coded for by the CD1D gene. The invention is based on the seminal discovery that targeting CD1d glycoproteins with NKT cell Th1 polarizing synthetic glycolipids can induce a potent innate and adaptive immune mediated tumor cell killing. Surprisingly, displacing tumor glycolipids bound to CD1d can redirect NKT cell response to a Th1 polarized response facilitating cancer immune surveillance and tumor cell death.

CD1d is assembled in the endoplasmic reticulum and complexes with β2-microglobulin. The CD1d glycoprotein/β2-microglobulin complex is stabilized by glycolipid binding to CD1d. While the hydrophobic portion of the glycolipid is buried deep within the CD hydrophobic binding groove, the polar portion of the glycolipid molecule, the sugar moiety, is exposed at the entrance of the groove for recognition by the NKT cell's TCR. Stabilization and orientation of the polar sugar moiety is achieved via interaction with polar amino acids located near the surface of the binding grove. Molecular dynamics simulation studies reveal that positioning of the polar sugar moiety and hydrogen bonding with the NKT cell TCR correlates to either a TH-1 or TH-2 polarized NKT cell response. Affinity and avidity of the glycolipid for CD1d stabilizes the CD1d/glycolipid/NKT cell TCR complex and appears to drive TH-1 polarization of the NKT cell response.

NKT cells also possess receptors for cytokines similar to innate cells such as NK and innate lymphoid cells. These cytokine receptors can be activated by steady state expression of certain inflammatory cytokines even in the absence of TCR signals. NKT cells can amalgamate signals from both TCR-mediated stimulations and inflammatory cytokines to manifest prompt release of an array of cytokines. These cytokines can in turn modulate different immune cells present in the tumor microenvironment (TME), thus influencing host immune responses to cancer. NKT cells' predominant tissue localization and ability to sense cancer-mediated changes in host lipid metabolism or breach in tissue integrity via recognition of endogenous lipids, makes NKT cells an ideal candidate for cancer immunotherapy.

Recent studies concerning NKT cell trafficking in cancer patients have demonstrated that NKT cells localize to primary and metastatic tumors in subsets of patients with common types of cancer, both in children and adults. Increased NKT cell invasion of tumor leads to better overall survival. Accumulating evidence also indicates that NKT cells target CD1d-positive tumor cells and CD1d-positive cells present in the tumor stroma in a CD1d-restricted manner. In the case of renal cell carcinoma, increasing CD1d-positive glycoprotein expression by tumor cells leads to poor overall patient survival.

NKT cells are known to express a chemokine receptor profile most similar to Th1 inflammatory homing cells. NKT cells have been shown to migrate toward neuroblastoma cells in a CCL2-dependent manner. In the tumor microenvironment, monocytes and tumor associated macrophages (TAMs) have been shown to up-regulate CCL20, a selective chemo-attractant for immature dendritic cells, effector-memory T cells, and NKT cells. TAMs contribute to tumor progression in various types of cancer, and are the dominant subset of CD45+ leukocytes that accumulate in human tumors and suppress immune cell function via multiple mechanisms. NKT cells help to mediate anti-tumor activity by removal of TAM-mediated growth support for tumor cells. TAMs cross-present glycolipids, and are killed by NKT cells in a CD1d-restricted manner.

T cells possess a diverse TCR repertoire to recognize several different peptide pathogens, allergens and cancer antigens resulting in a precursor frequency of epitope-specific CD8+ T cells that is extremely low in number ( 1/100000- 1/1000000 of the total T cell population), and require 7-14 days to respond to antigen stimulation. Due to the invariant nature of the NKT cell TCR which recognizes structurally diverse glycolipid antigens presented in the context of the non-polymorphic CD1d, NKT cell numbers are significantly higher ( 1/100- 1/5000 of the total T cell population), and NKT cells respond instantly or within a few hours of antigen stimulation. In addition to circulating within the periphery, NKT cells are tissue-resident lymphocytes found in lymph, spleen, lung, epithelial lining of the GI tract, liver and thymus.

Glycolipids are complex molecules consisting of a ceramide lipid moiety linked to a glycan chain of variable length and structure. Among these are found the gangliosides, which are sialylated glycolipids ubiquitously distributed on the outer layer of vertebrate plasma membranes. Changes in the expression of certain species of gangliosides have been described to occur during cell proliferation, differentiation, and ontogenesis. Aberrant and elevated expression of gangliosides has been also observed in different types of cancer cells, thereby promoting tumor survival. Moreover, gangliosides are actively released from the membrane of tumor cells, having a strong impact on impairing anti-tumor immunity. Tumors shed gangliosides into the extracellular milieu where they are in dynamic equilibrium between monomeric, multimeric, and larger hetero-complexed forms. From these various states, they transfer to different immune cells, modify their membrane composition, and induce modifications that modulate innate and adaptive immunity. Cancer cells shed glycolipid gangliosides which bind to CD1d glycoprotein causing NKT cells to shift to a TH-2 response polarization allowing cancers to evade destruction by the immune system, or by completely masking the ability of the tumors to be recognized by NKT cells. NKT cells are known to invade tumors such as ovarian cancer tissue. Tumors are known to shed glycolipid gangliosides which cause immune system dysregulation allowing cancer cells to escape immune destruction—a type of cancer immuno-editing. Ovarian cancer cells shed Ganglioside D3 (GD3), and demonstrated that GD3 inhibited NKT cell function.

The present invention illustrates that certain glycolipids, such as OT-0010, reverses CD1d/GD3 ganglioside/NKT cell blockade providing restored immune surveillance and tumor cell destruction.

As used herein, a “therapeutically effective amount” of a compound, is intended to qualify the amount of active ingredients used in the treatment of a disease or disorder. This amount will achieve the goal of reducing or eliminating the said disease or disorder. The exact dosage and frequency of administration depends on the particular compound of the invention used, the particular condition being treated, the severity of the condition being treated, the age, weight and general physical condition of the particular subject as well as the other medication, the patient may be taking, as is well known to those skilled in the art. Furthermore, said “therapeutically effective amount” may be lowered or increased depending on the response of the treated subject and/or depending on the evaluation of the physician prescribing the compounds of the instant invention.

For example, as used herein, a therapeutically effective amount is an amount of glycolipid effective in stimulating a shift of natural killer (NK) cells to a Th1 immune response. For example, the glycolipid has a human CD1d EC50 binding affinity of at least about 5× less than the corresponding tumor-specific glycolipid EC50 binding affinity to human CD1d, and results in an increase in interferon-gamma production of at least about 2-fold within about 20 hours of administration, as compared with levels of interferon-gamma production prior to administration.

As used herein, reference to “treating” or “treatment” of a subject is intended to include prophylaxis. The term “subject” means all mammals including humans. Examples of subjects include humans, cows, dogs, cats, goats, sheep, pigs, and rabbits. Preferably, the subject is a human.

In addition to invention compounds, one of skill in the art would recognize that other therapeutic compounds including chemotherapeutic agents, anti-inflammatory agents, and therapeutic antibodies can be used prior to, simultaneously with or following treatment with invention compounds. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthalene acetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-α, etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8 gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-α inhibitors such as tenidap, anti-TNF antibodies or soluble TNF receptor, and rapamycin (sirolimus or Rapamune) or derivatives thereof.

Other agents that may be administered in combination with invention compositions and methods include protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

As used herein, the term “cancer” or “cancerous growth” means the uncontrolled, abnormal growth of cells and includes within its scope all the well-known diseases that are caused by the uncontrolled and abnormal growth of cells. Non-limiting examples of common cancers include bladder cancer, breast cancer, ovarian cancer, pancreatic cancer, and gastric cancer, cervical cancer, colon cancer, endometrial cancer, head and neck cancer, lung cancer, melanoma, multiple myeloma, leukemia (e.g., myeloid, lymphocytic, myelocytic and lymphoblastic leukemias), non-hodgkin's lymphoma, prostate cancer, rectal cancer, and malignant melanomas.

The term “alkenyl” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like.

The term “amido” as used herein, alone or in combination, refers to an amino group as described below attached to the parent molecular moiety through a carbonyl group, or vice versa. The term “acetamido” refers to the group —NHC(═O)CH₃.

The term “halogen” as used herein, alone or in combination, refers to fluorine, chlorine, bromine, or iodine.

The term “nitro” as used herein, alone or in combination, refers to —NO₂.

The terms “oxy” or “oxa” as used herein, alone or in combination, refer to —O—.

The term “phenyl” refers to the group C₆H₅—.

The term “furan” refers to a heterocyclic organic compound, consisting of a five-membered aromatic ring with four carbon atoms and one oxygen.

The term “thio” as used herein, alone or in combination, refers to a —S— group or an ether wherein the oxygen is replaced with sulfur. The oxidized derivatives of the thio group, namely sulfinyl and sulfonyl, are included in the definition of thio.

Asymmetric centers exist in the compounds of the present invention. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers, including keto-enol tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002), the entire contents of which are herein incorporated by reference.

The term “therapeutically acceptable salt” or “pharmaceutically acceptable salt” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds of the present invention, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences, the entire contents of which are incorporated herein by reference. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Before the present compositions and methods are described, it is to be understood that this invention is not limited to particular methods, compositions, and experimental conditions described, as such methods, compositions, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. The definitions set forth below are for understanding of the disclosure but shall in no way be considered to supplant the understanding of the terms held by those of ordinary skill in the art.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

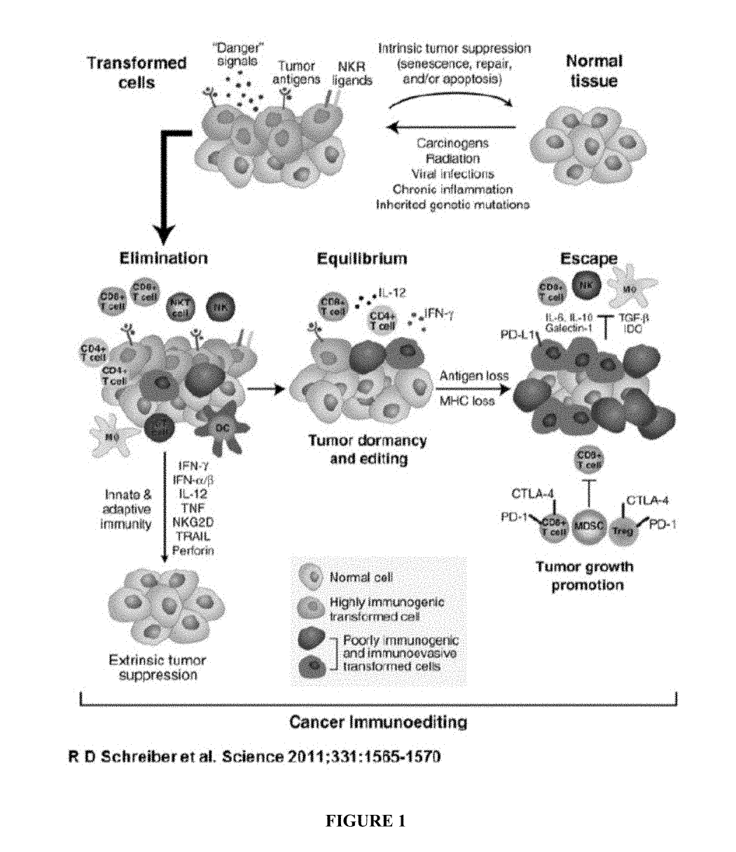

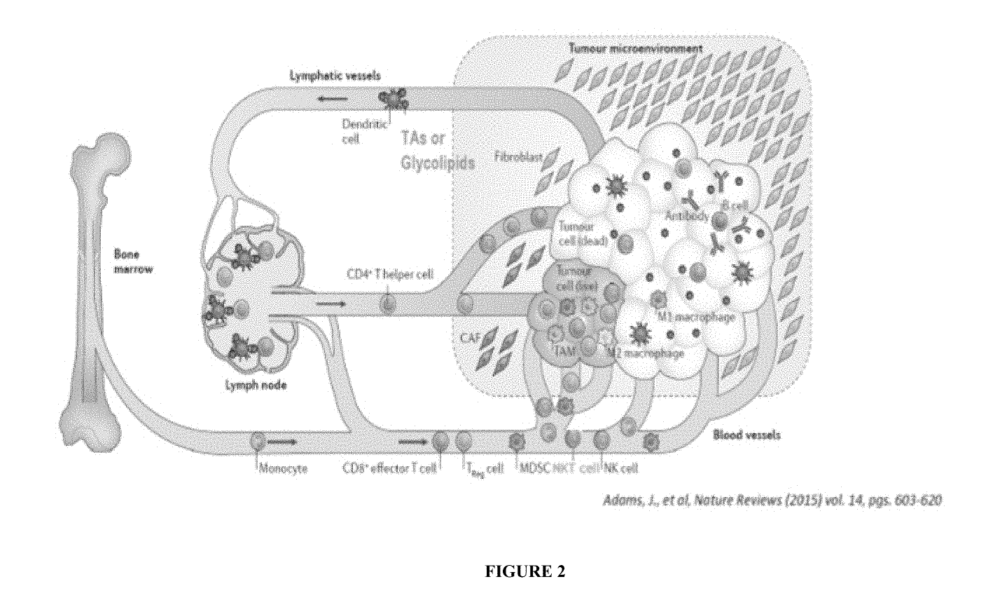

FIG. 1 outlines how cancer evades immune surveillance. The present invention is directed to methods that are directed to either an immuno-oncology (I-O) monotherapy strategy or to a combination I-O therapeutic agent strategy which relieve a specific immune checkpoint blockade as a means to fight cancer (FIG. 2).

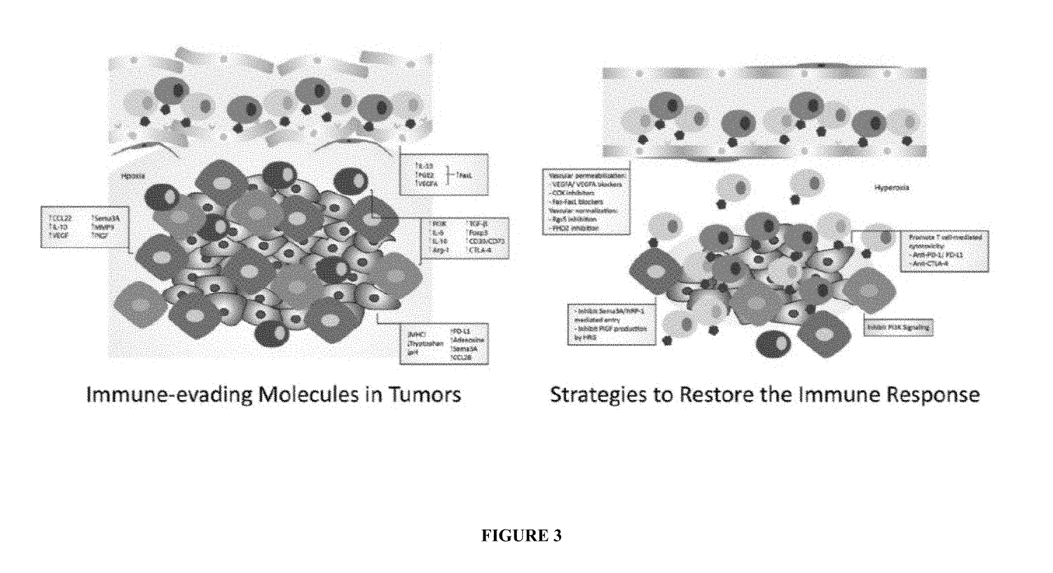

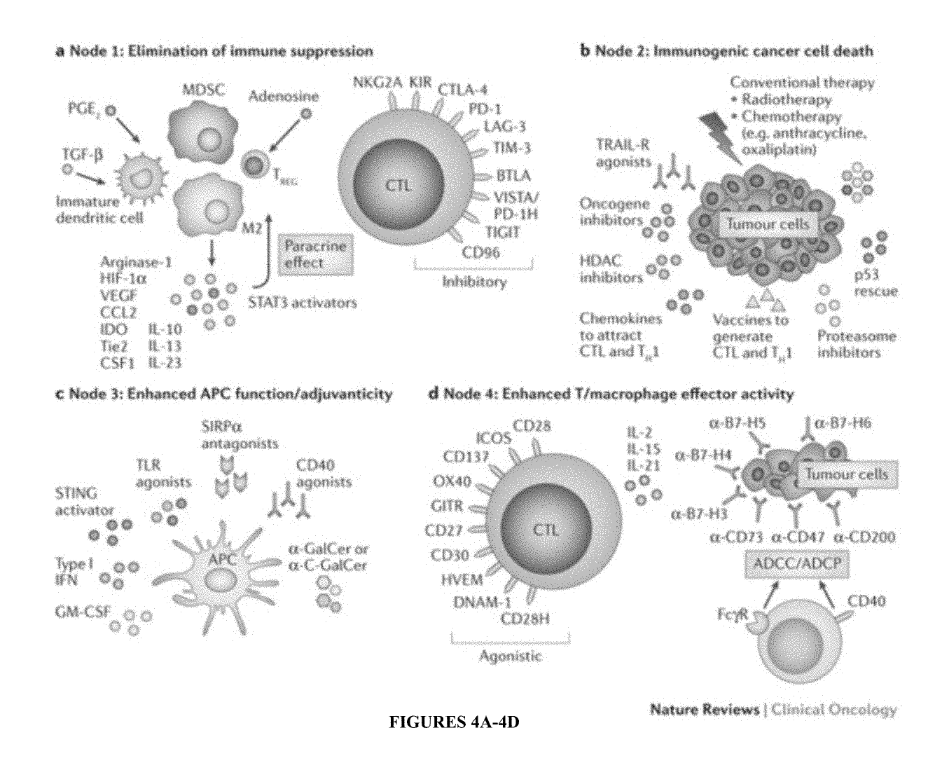

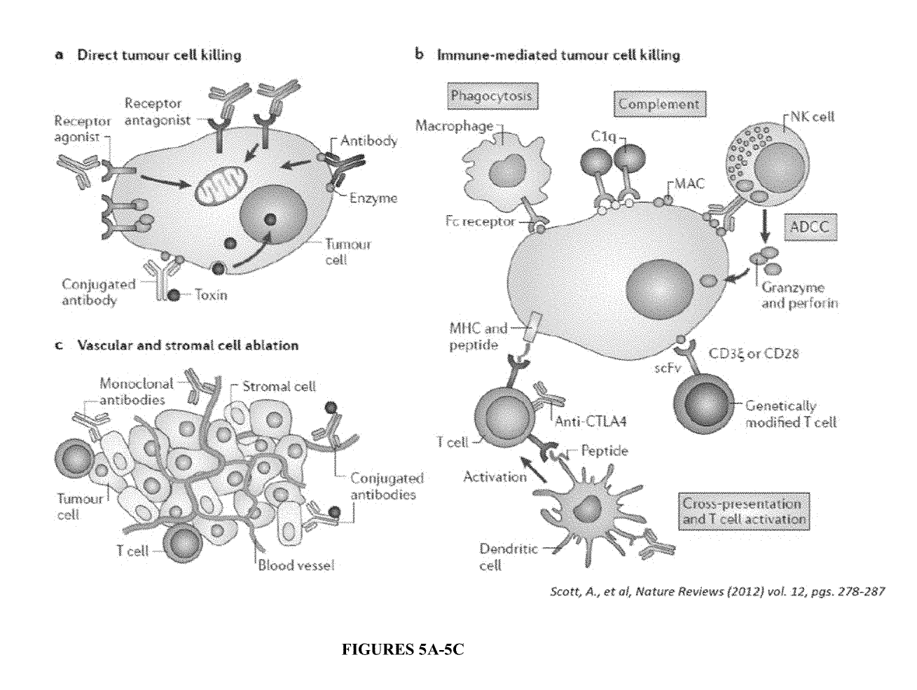

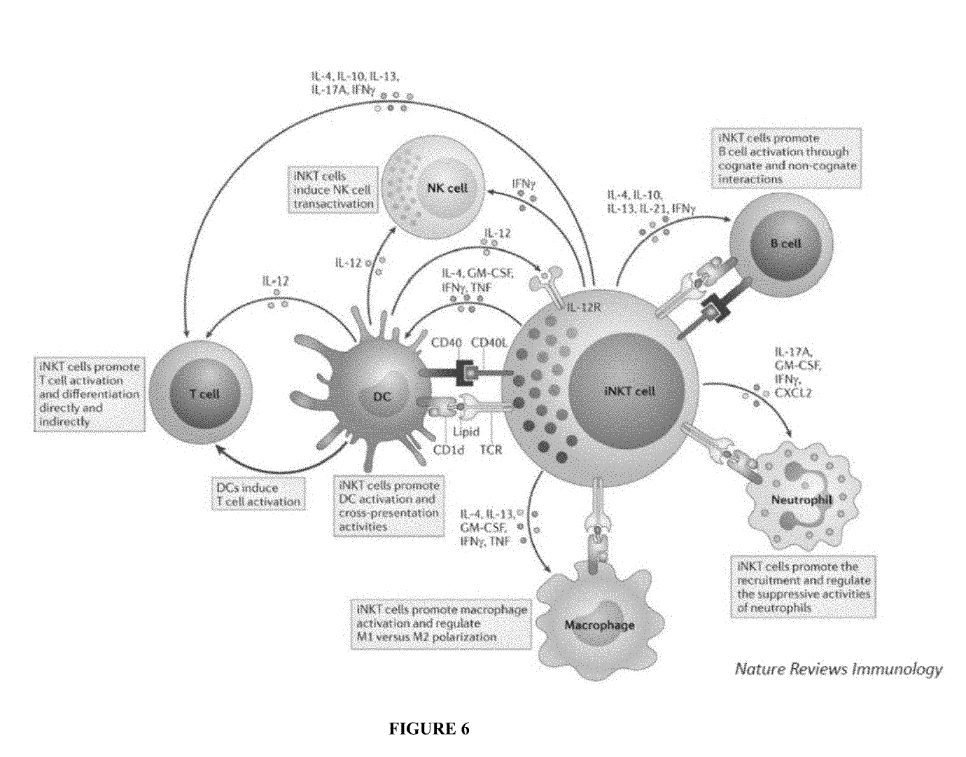

A goal of these methods is to restore immune protection (FIG. 3). Key nodes of the immune system to target with a combination immune-oncology therapeutics are outlined in FIG. 4. Antibody killing mechanisms are outlined in FIG. 5. An overview of targeting natural killer T-cells (NKT cells) to relieve immune system checkpoint blockade is presented in FIG. 6.

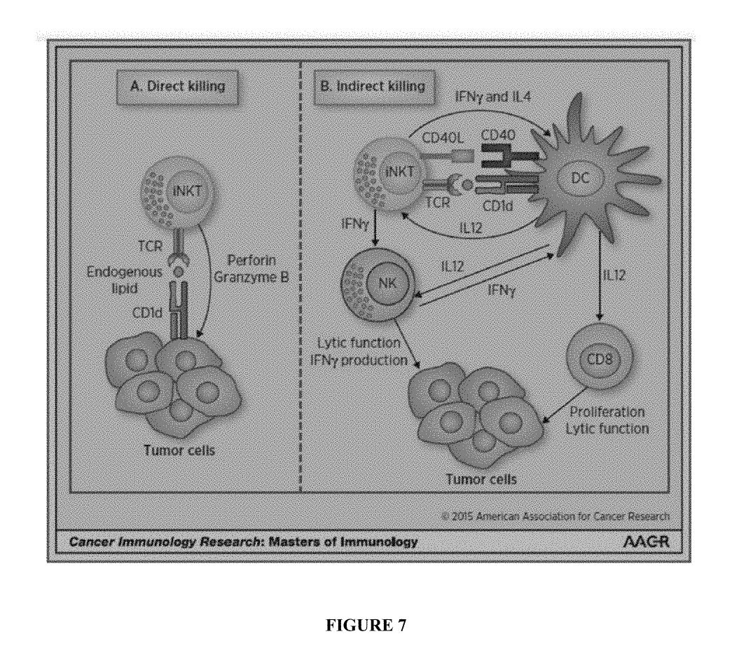

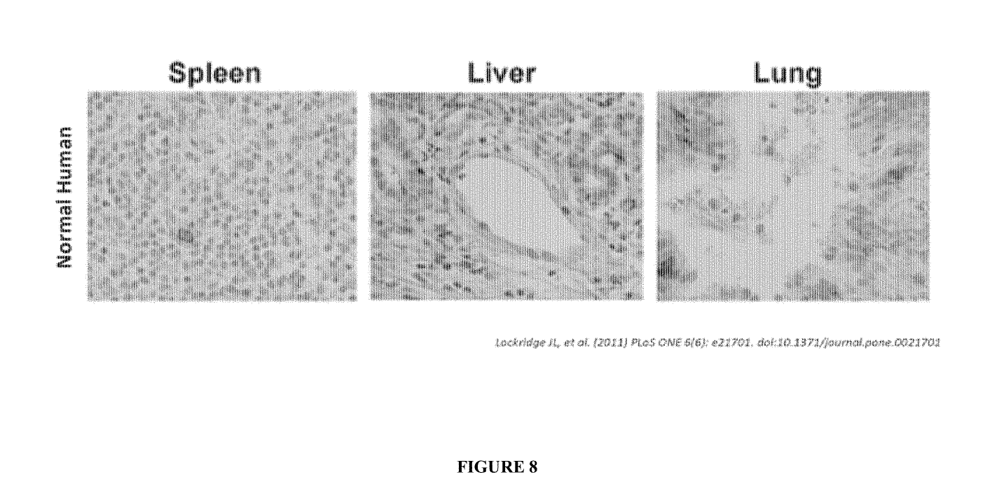

NKT cells kill tumor cells expressing CD1d or kill tumor cells by interacting with immune cell partners (FIG. 7). CD1d is expressed in human tissue and also in certain tumor types, such as hematopoietic malignancies, prostate cancers, renal cancers, breast cancer, ovarian cancer, multiple myeloma, melanoma, colon cancer, lung cancer, cervical cancer, and some neurological tumors (FIG. 8).

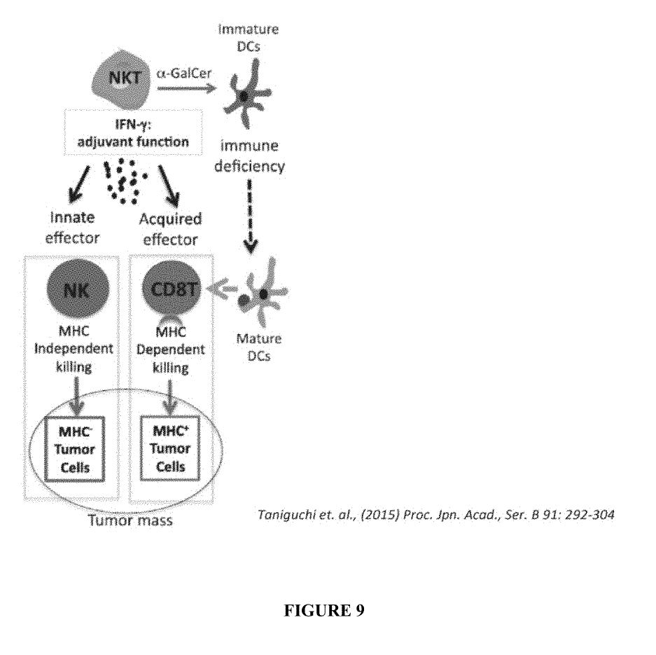

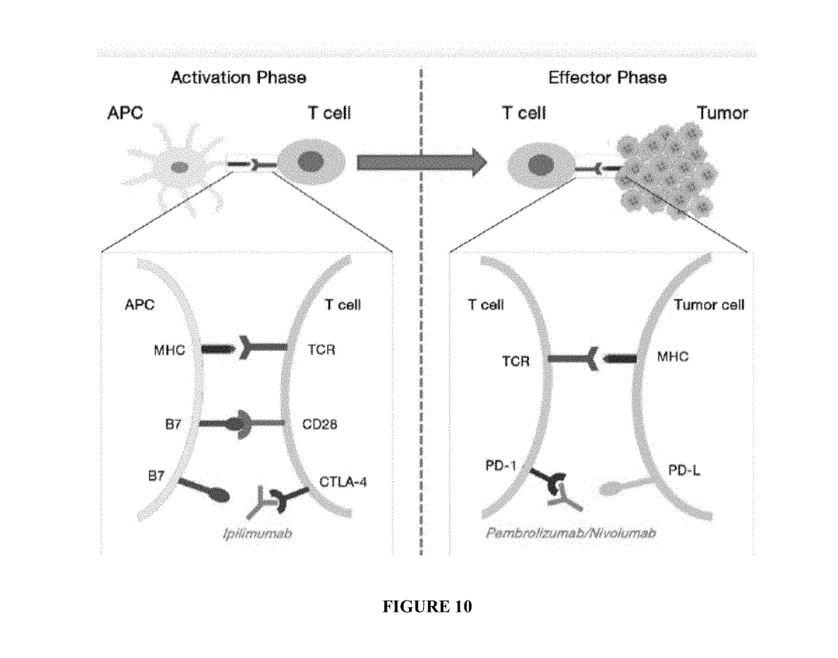

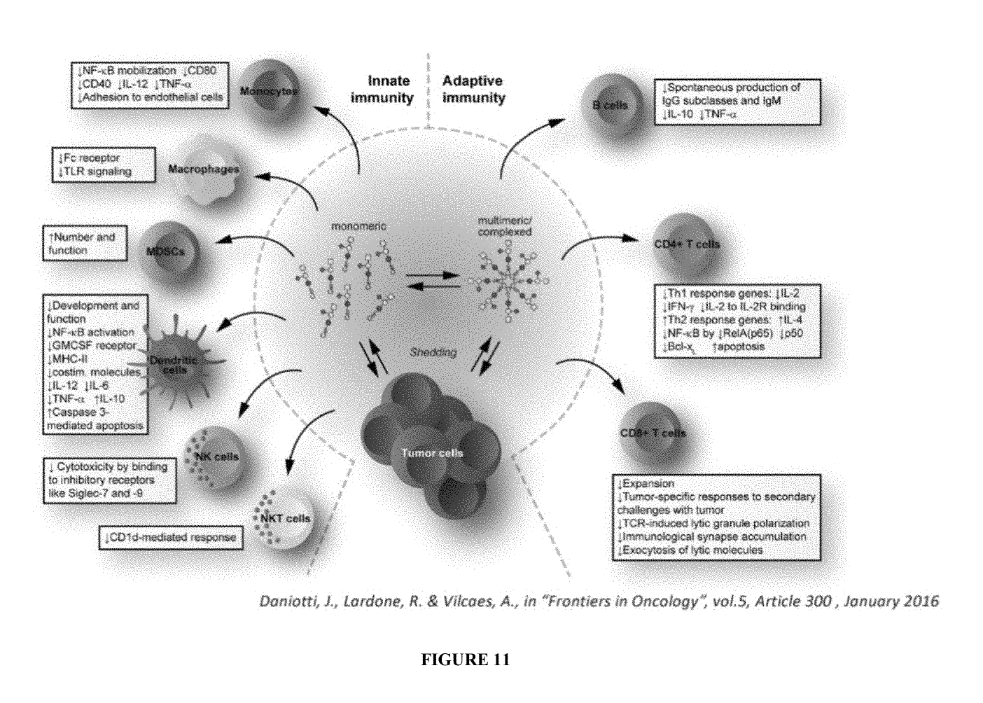

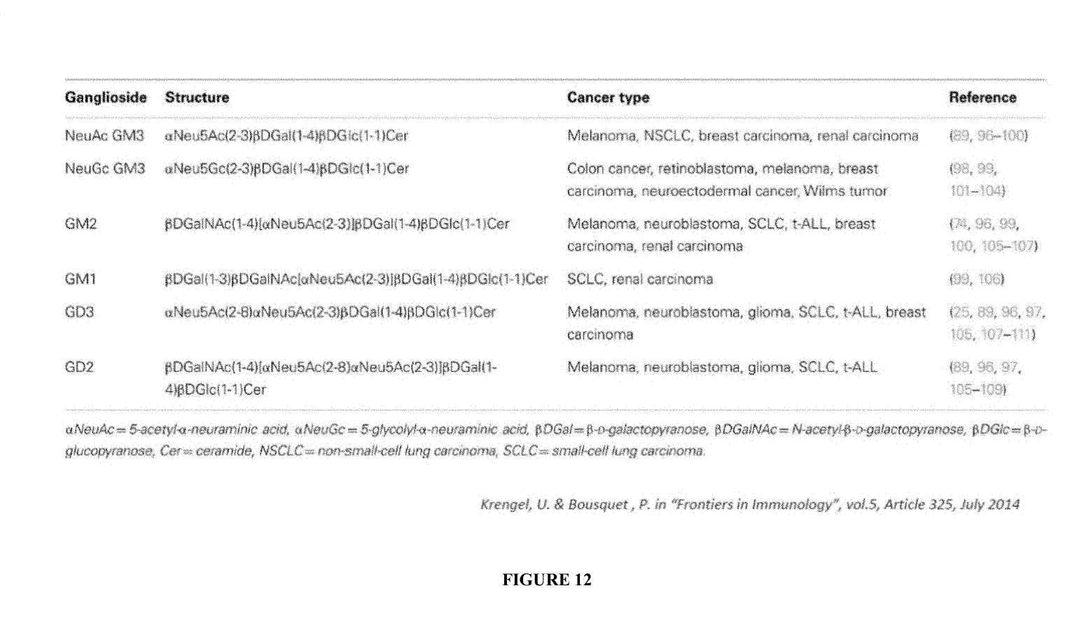

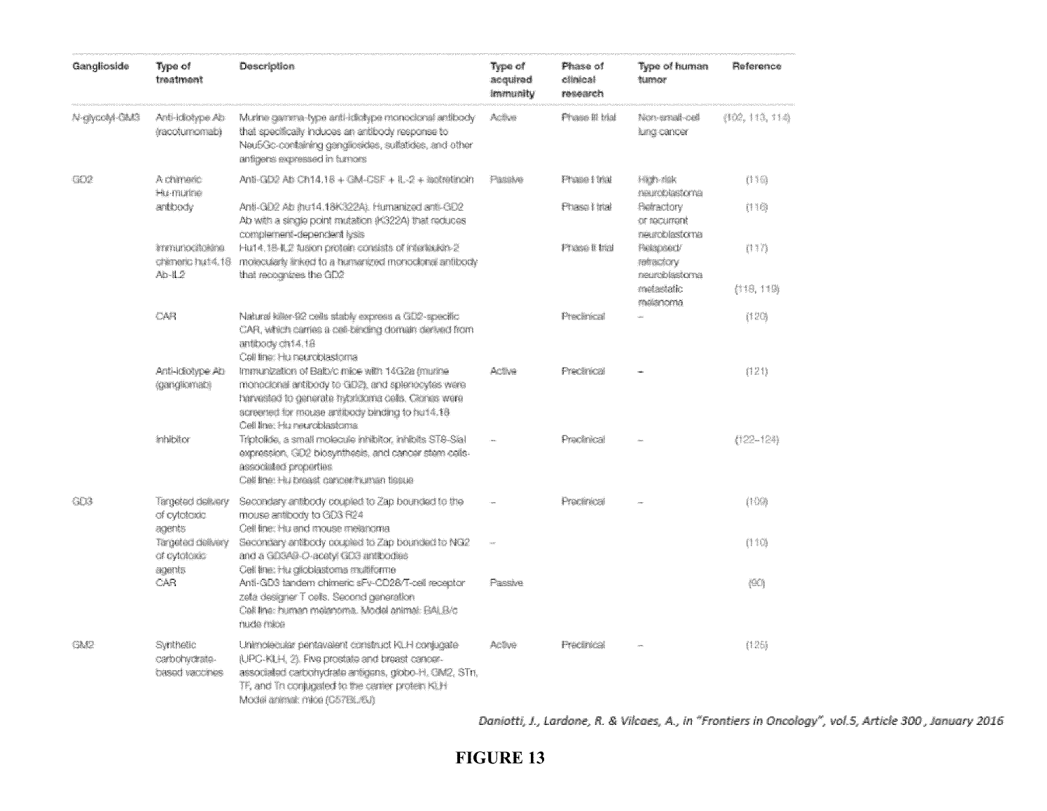

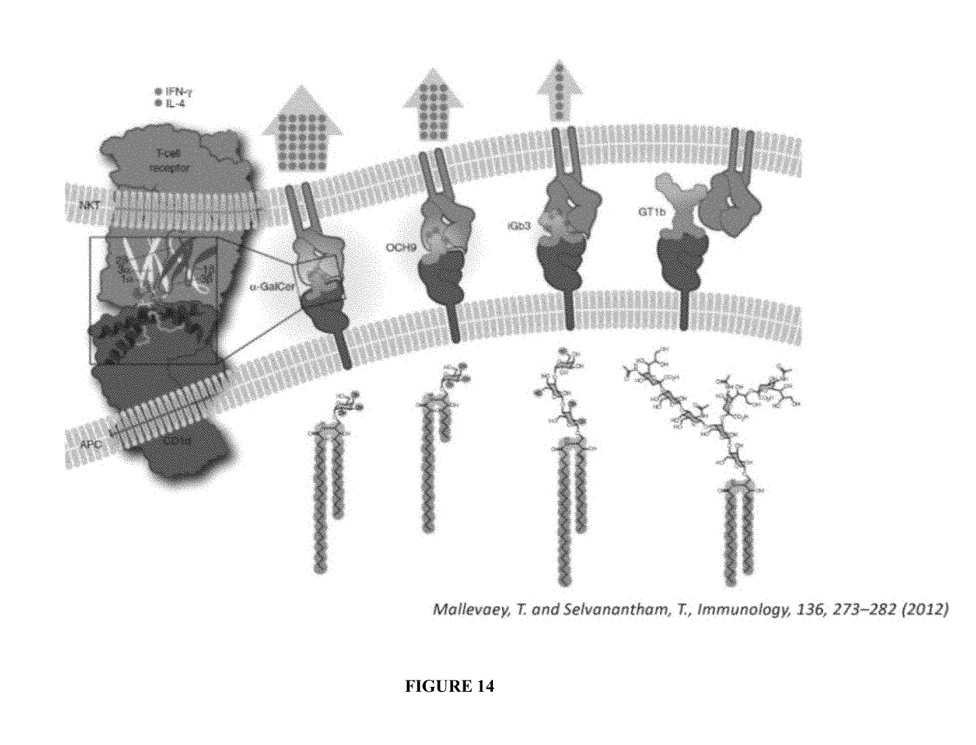

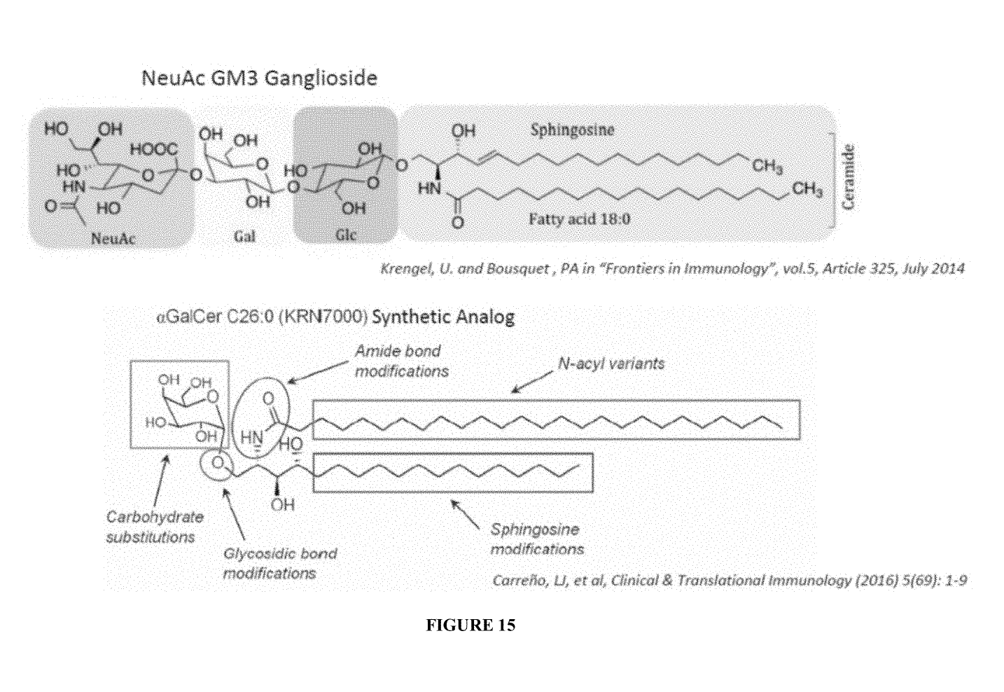

FIG. 9 discloses an overview of how NKT cells interact with immature dendritic cells in the presence of glycolipids. FIG. 10 shows current immune checkpoint therapeutic strategy. FIGS. 11-16 disclose aspects of glycolipids and glycolipid gangliosides as they relate to the immune system and cancer.

Immune responses may be broadly divided into Cell Mediated Immunity (CMI) and Humoral Immunity (HI). CMI is associated with Th1 CD4+T lymphocytes producing cytokines IL-2, interferon (IFN) and tumor necrosis factor (TNF). Immune Th1 responses results in tumor rejection as Th1 pathways typically produce activation of cytotoxic T-cell lymphocytes (CTL), natural killer T (NKT) cells, natural killer (NK) cells, macrophages and monocytes—all of which can attack cancer cells and generally defend against tumors. In contrast, HI is associated with Th2 CD4+T lymphocytes producing IL-4, IL-6 and IL-10. Deviation towards Th2 response prevents tumor rejection. Almost all malignancies are associated with suppression of the CMI.

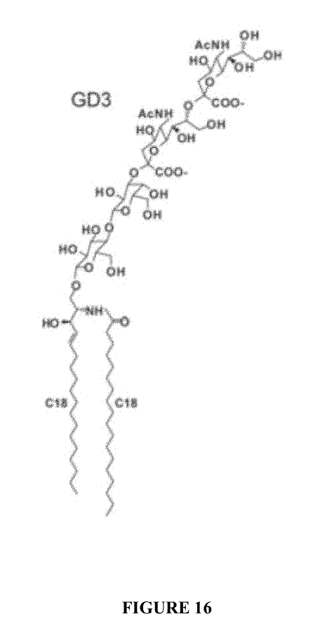

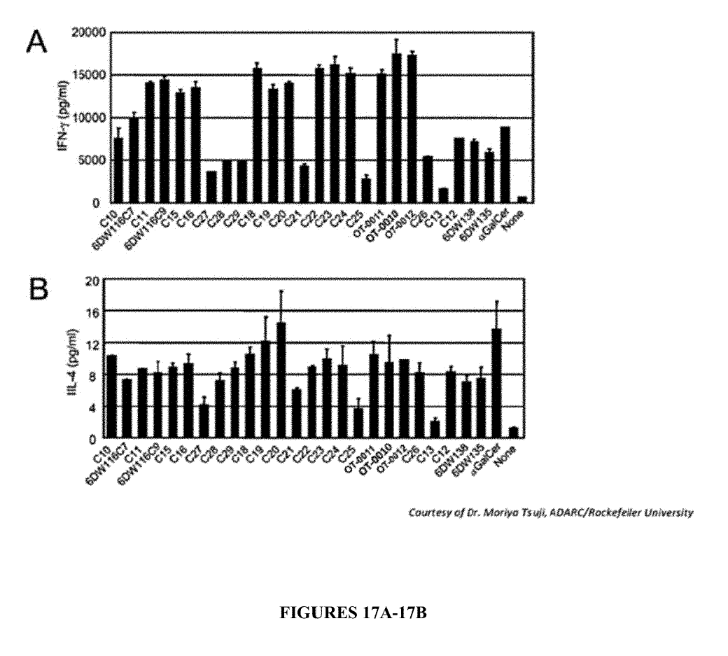

The GD3 ganglioside is a tumor-specific glycolipid antigen in humans (FIG. 16). GD3 is overexpressed on virtually all melanomas as well as several other tumors such as renal cell carcinoma and prostate cancer. GD3 is shed into the microtumor environment by tumors. GD3 binds to CD1d and shifts NKT cells from a Th1 response to a Th2 response. FIG. 17A-B illustrate the NKT cell response to OT-0010 glycolipid presented by HeLa-CD1d cancer cell.

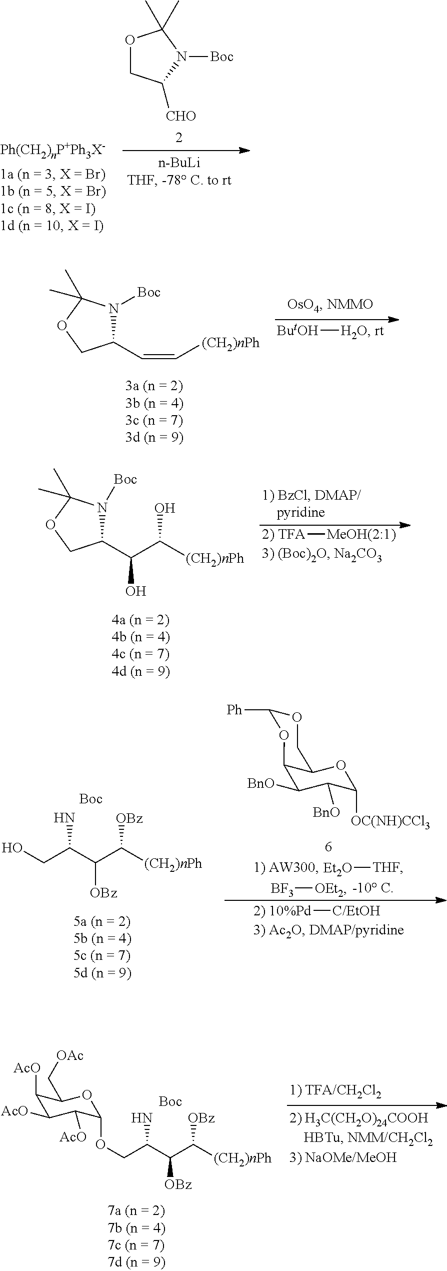

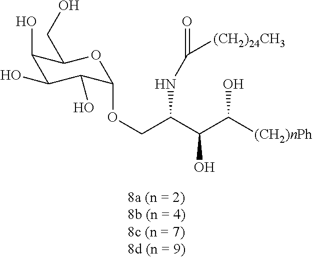

The above scheme illustrates the synthesis of α-GalCer analogues. Phytosphingosine chain analogues were synthesized according to the scheme described. Garner aldehyde 2 was coupled with Wittig reagents 1a-1d to give corresponding olefins 3a-3d. Compounds 3a-3d were treated with osmium tetroxide to give diols 4a-4d and an undesired isomer. The hydroxyl groups of the diol were protected with benzoyl groups and the isopropylidene group removed by trifluoacetic acid (TFA). After reprotection of the amine with tert-butoxycarbonyl (Boc) anhydride, the phytosphingosine analogues were glycosylated to yield mainly the α-linked product. After a series of protecting group manipulations, hexacosanoic acid was coupled to the free amine in the presence of 2-(1H-benzotriazol-1-yl)-1,1,3,3-tetramethyluronium hexafluorophosphate (HBTu). Deprotection of galactose protecting groups using 20% Pd(OH)2 and deprotection of benzoyl groups using NaOMe afforded the target compounds.

TABLE 1
Structure of α-GalCer analogues.

A

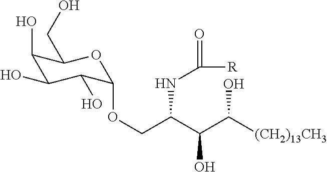

B

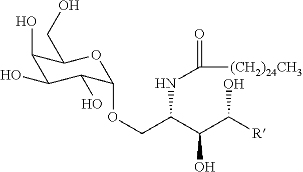

| Glycolipid | Structure |
|---|---|
| α-GalCer | A. R = (CH₂)₂₄CH₃ |
| C10 | A. R = (CH₂)₅Ph |
| 6DW116C7 | A. R = (CH₂)₆Ph |
| C11 | A. R = (CH₂)₇Ph |
| 6DW116C9 | A. R = (CH₂)₈Ph |
| C15 | A. R = (CH₂)₉Ph |
| C16 (Formula V) | A. R = (CH₂)₁₀Ph |
| C27 | A. R = (CH₂)₁₄Ph |
| C28 | A. R = (CH₂)₂₀Ph |
| C29 | A. R = (CH₂)₂₄Ph |
| C18 | A. R = (CH₂)₅Ph(p-OMe) |
| C19 | A. R = (CH₂)₅Ph(p-F) |
| C20 | A. R = (CH₂)₅Ph(p-CF₃) |
| C21 | A. R = (CH₂)₅Ph(p-Ph) |
| C22 | A. R = (CH₂)₇Ph(p-OMe) |
| C23 | A. R = (CH₂)₇Ph(p-F) |
| C24 | A. R = (CH₂)₇Ph(p-CF₃) |
| C25 | A. R = (CH₂)₇Ph(p-Ph) |
| OT-0011 (Formula II) | A. R = (CH₂)₁₀Ph(p-OMe) |
| OT-0010 (Formula III) | A. R = (CH₂)₁₀Ph(p-F) |
| OT-0012 (Formula IV) | A. R = (CH₂)₁₀Ph(p-CF₃) |
| C26 | A. R = (CH₂)₁₀Ph(p-Ph) |
| C13 | B. R′ = (CH₂)₂Ph |
| C12 | B. R′ = (CH₂)₄Ph |
| 6DW138 | B. R′ = (CH₂)₇Ph |
| 6DW135 | B. R′ = (CH₂)₉Ph |

Following the above synthetic scheme, a library of α-GalCer analogues are synthesized and shown in Table 1. Analogues in group A have a modification at a fatty acyl chain, whereas analogues in group B have a terminal benzene ring at phytosphingosine chain. Further details of the synthesis and characterization of this library of α-GalCer analogues can be found in Li et al. (2010) Proc. Natl. Acad. Sci. U.S.A. 107:13010-15, herein incorporated by reference in its entirety.

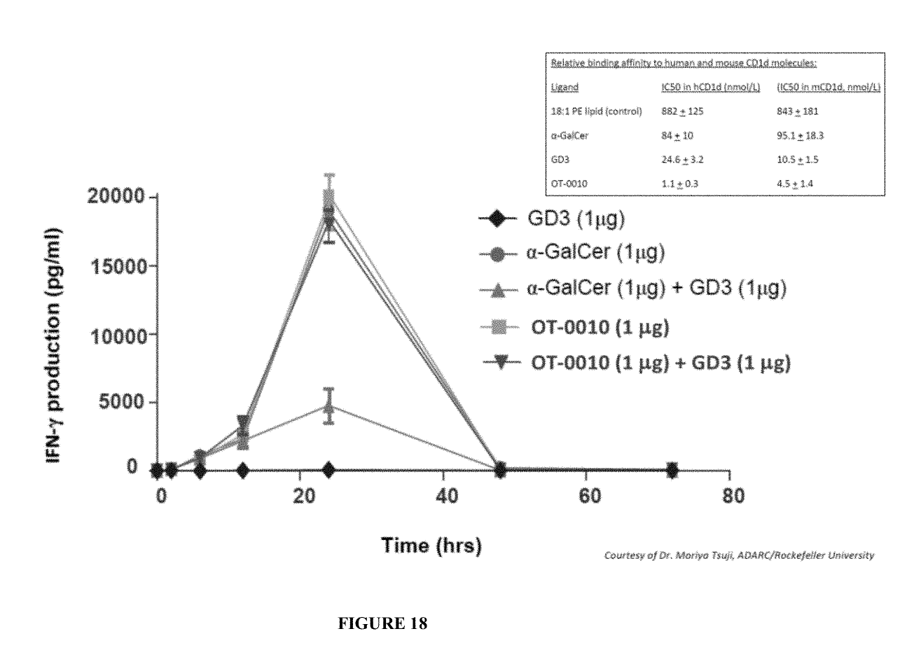

FIG. 18 shows that OT-0010 reverses GD3 ganglioside immune blockade by shifting NKT cells to a Th1 response. OT-0010 is effective at restoring NKT cell function when wild-type C57BL/6 mice were injected intravenously with either αGalCer (KRN7000) or OT-0010 in the presence or absence of GD3. Inhibition of IFNγ production was observed in GD3 alone and αGalCer (KRN7000)+GD3 treated mice. Inhibition of IFNγ production was the result of CD1d-dependent dendritic cell presentation of GD3 to NKT cells. Mice administered OT-0010 or OT-0010+GD3 showed no decrease in IFNγ production due to the higher binding affinity of OT-0010 to CD1d compared to GD3.

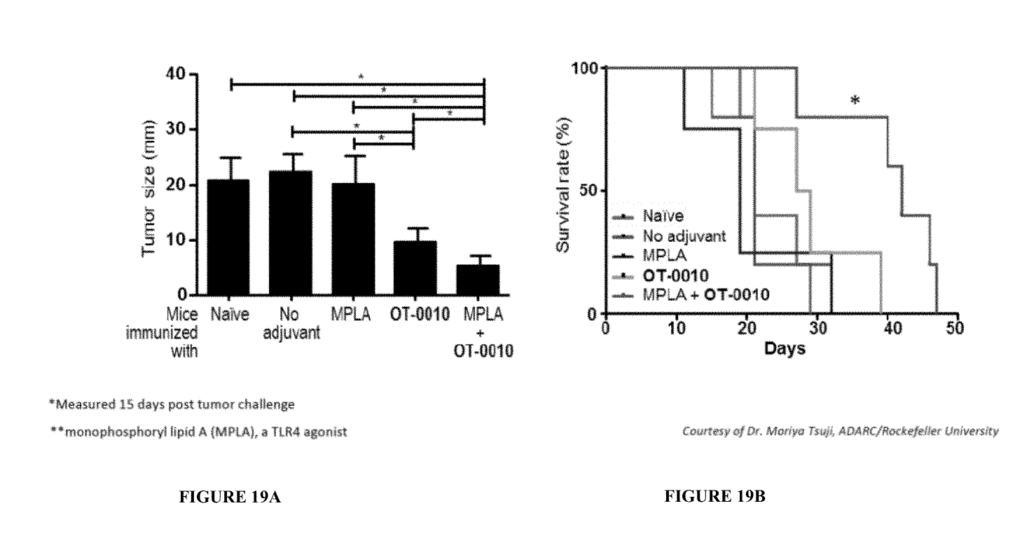

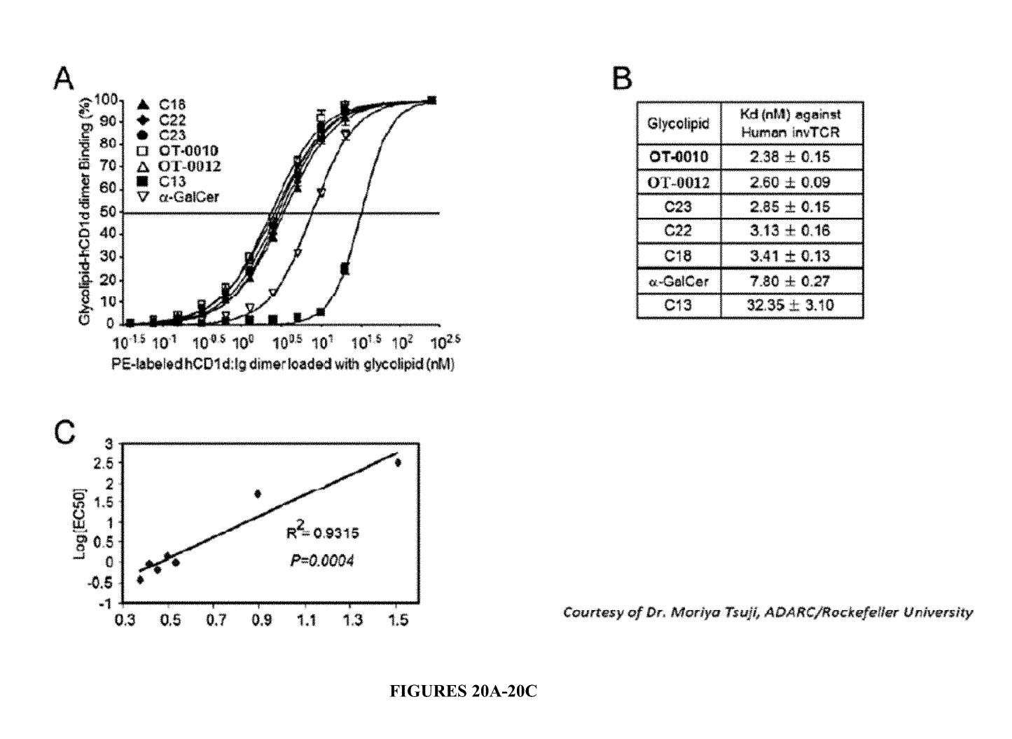

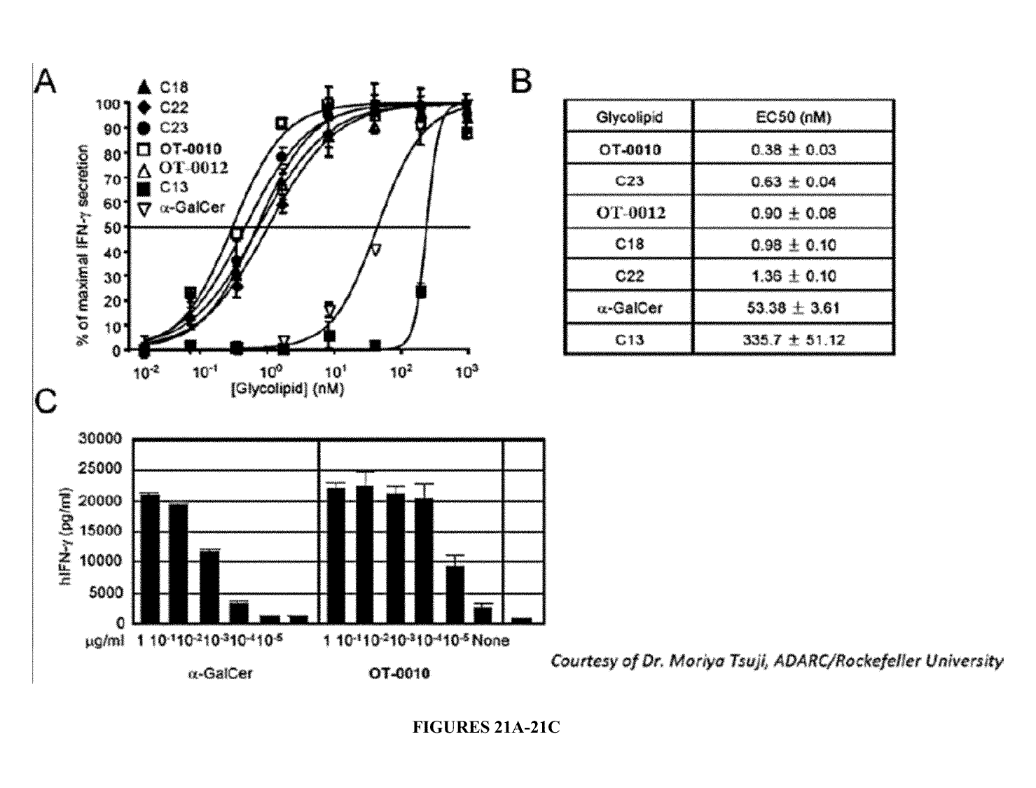

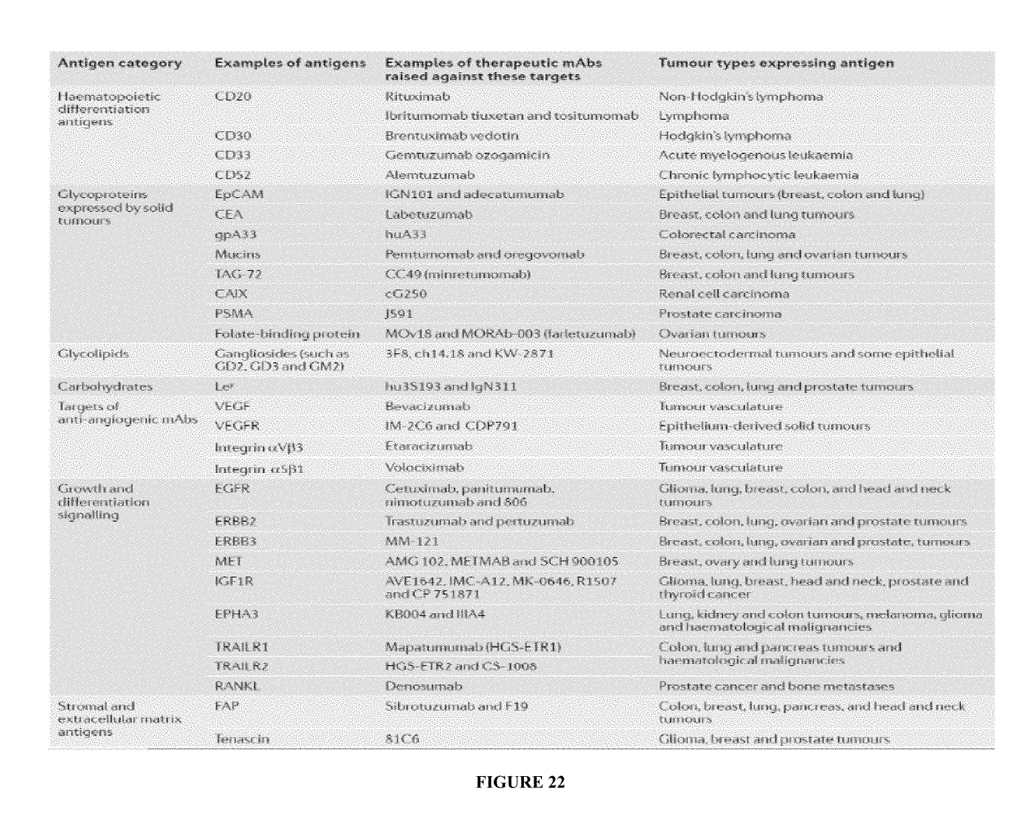

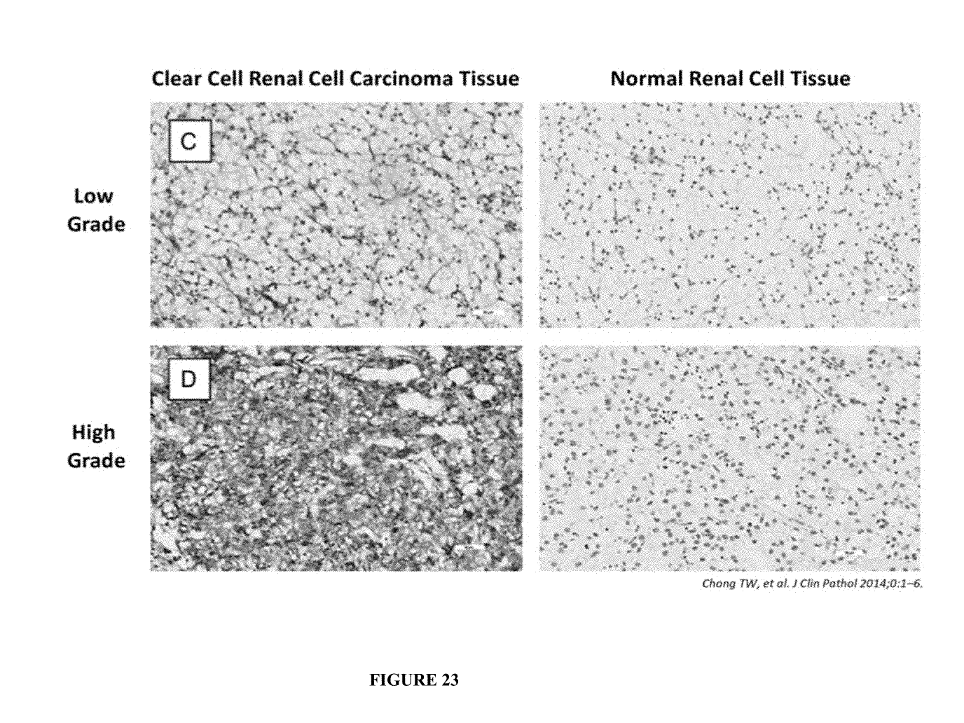

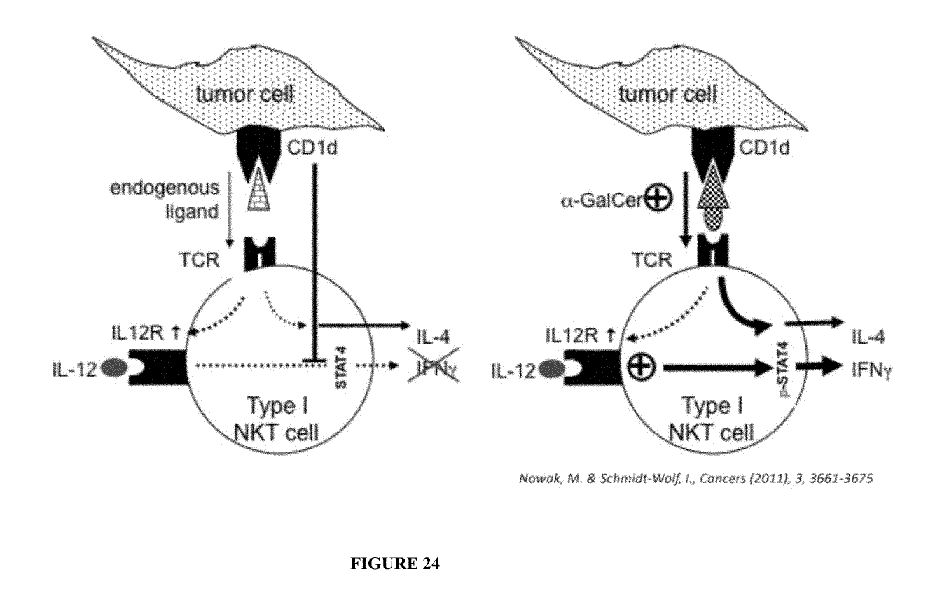

FIG. 19A-B shows OT-0010 combination immuno-oncology therapeutic tumor challenge mouse model. FIG. 20A-B shows OT-0010 human NKT cell TCR binding. FIG. 21A-C shows OT-0010 human IFN-γ production. FIG. 22 illustrates tumor-associated antigens targeted by therapeutic antibodies. FIG. 23 shows that clear renal cell carcinoma over expresses CD1d in a disease dependent manner. FIG. 24 illustrates how NKT cells interact directly with prostate cancer cells via CD1d.

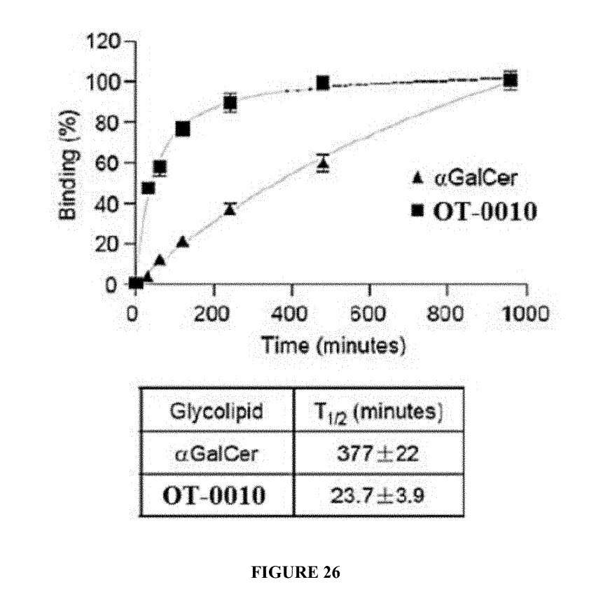

FIG. 26 shows that CD1d captures OT-0010 faster than α-GalCer, which shows the enhanced avidity of OT-0010 compared α-GalCer. Thus, FIG. 18 above shows that OT-0010 has higher affinity for CD1d which in biochemistry terms is defined as the single interaction, here in FIG. 26, OT-0010 also has higher avidity which in biochemistry terms is defined as the sum of all of the interactions between CD1d, OT-0010 and the TCR of the NKT cell. The increased affinity and avidity of OT-0010 binding to CD1d compared to GD3 and other tumor-shed gangliosides make it a promising pharmaceutical agent to shift NKT cell from a Th2 immune response to a Th1 response.

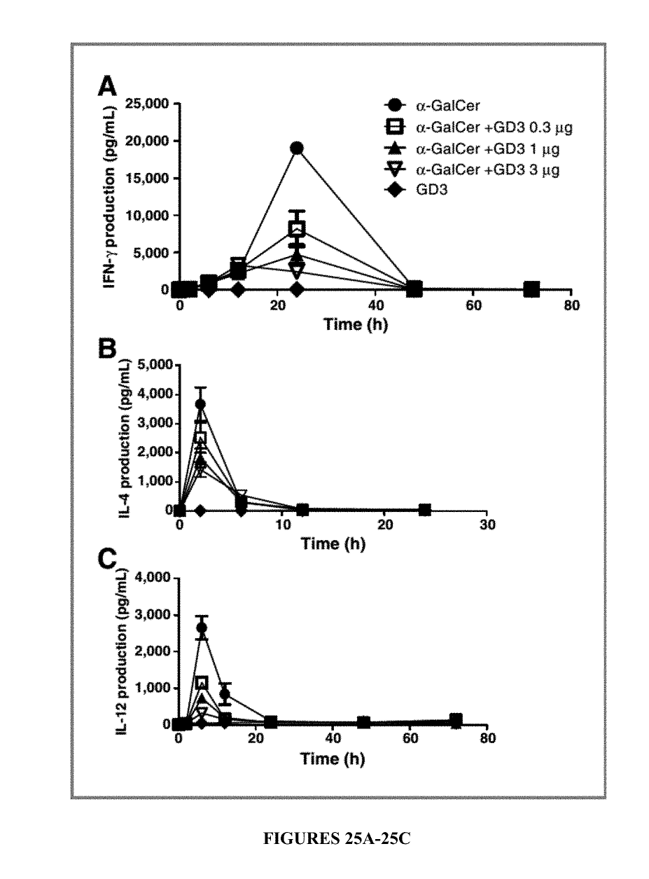

From FIG. 25, it is evident that GD3 causes a dramatic decrease in IFN-γ production by NKT cells which is indicative of shifting NKT cell from a Th1 response to a Th2 response. It is further evident that α-GalCer does not restore IFN-γ levels in the presence of GD3. This failure to restore IFN-γ levels indicates that NKT cells are not fully restored to a Th1 response profile, but are still in a Th2 response profile, hence suppressed by the endogenously shed GD3 tumor glycolipid. Manolova suggests single-chain glycolipids for CD1b ligand exchange (Manolova et al. (2002) Comptes Rendus de l'Academie Bulgare des Sciences 55:69-74). However, the inventors of the instant disclosure discovered that glycolipids with two fatty acid hydrophobic chains exhibit surprisingly strong binding affinity to CD1d. In addition, the glycolipids of the present invention are able to fully restore IFN-γ levels, and cause a restoration of NKT cells to a Th-1 response profile (FIG. 18). Surprisingly, a low concentration of OT-0010 of only 0.1 μgm restores 100% NKT cell function as evidenced by the IFN-γ secretion. When α-GalCer is administered at a dosage of 3 μgm, only 50% NKT cell function is restored (FIG. 25). While not wishing to be bound by theory, the reason why the glycolipids of the present invention are able to restore NKT cells to a Th-1 response profile is because these glycolipids bind more strongly to CD1d. From a thermodynamic perspective, it is impossible for GD3 to displace these glycolipids from CD1d. Referring to the insert on “Relative binding affinity” in FIG. 18, OT-0010 (Formula III) binds 24× more strongly to CD1d compared to GD3, and 84× more strongly compared to α-GalCer. Again, while not wishing to be bound by theory, it is believed that the glycolipids of the present invention bind more strongly to CD1d due to the incorporation of the -phenyl ring to the distal end of the aliphatic carbon chain attached to the amide group connected to the C1 glycoside linkage of the galactose sugar moiety. The terminal fluorine, —OMe, or —CF₃ further enhance the binding of the glycolipids of the present invention to CD1d. The results disclosed in FIG. 18 are unexpected. Prior to the inventors' discovery, there would have been no reason for one of ordinary skill in the art to test a glycolipid which has a superior binding affinity for human CD1d to see if the endogenously shed GD3 tumor glycolipid would be displaced, and IFN-γ levels would be restored indicating the Th2 to Th1 shift by NKT cells.

### EXAMPLES

### Example 1

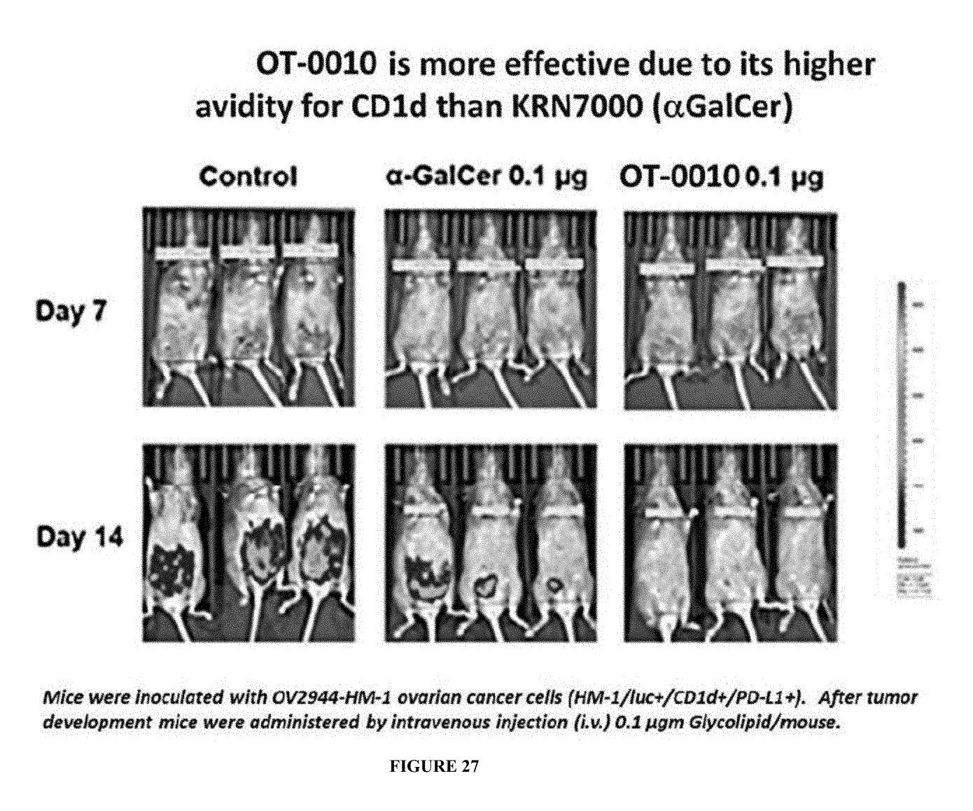

OT-0010 has demonstrated superior NKT cell activation leading to potent tumor cell killing in a variety of in vivo tumor models. FIG. 27 shows OT-0010 dose escalation study with OV2944-HM-1 ovarian cancer cell (HM-1/luc+/CD1d+/PD-L1+) in WT C57BL/6 mice. After tumor development mice were administered by intravenous injection at 0.1 μgm glycolipid/mouse. OT-0010 is more effective due to its higher avidity for CD1d than KRN7000 (α-GalCer).

### Example 2

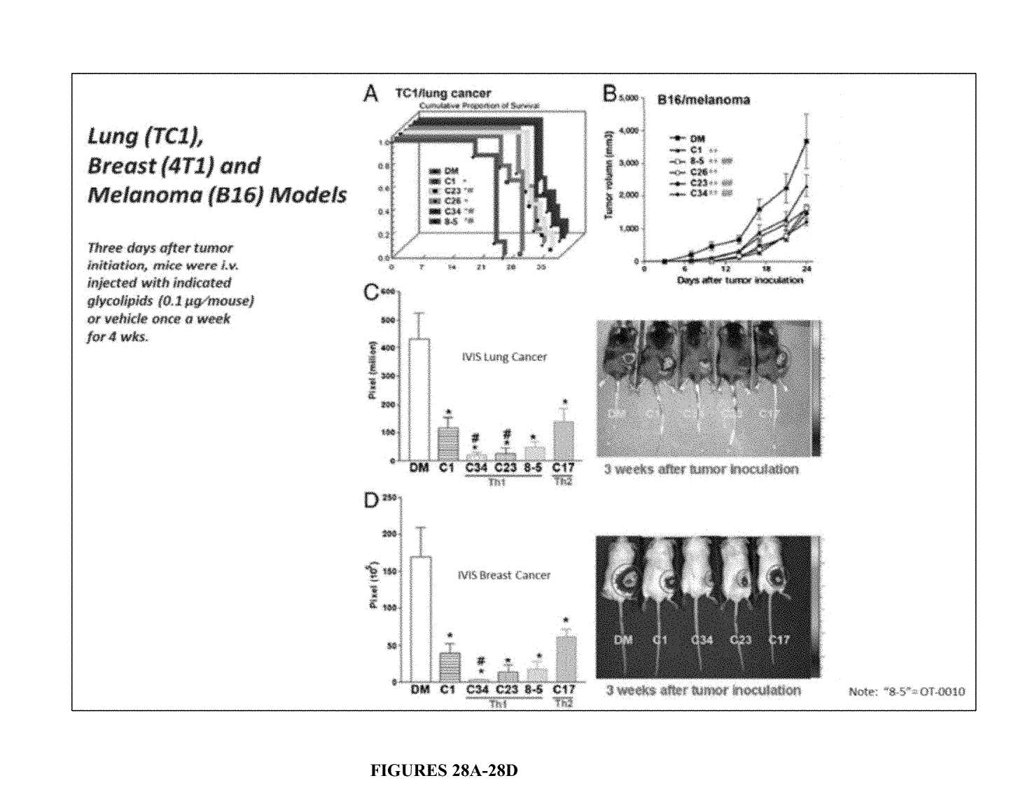

FIG. 28A-D shows that OT-0010 improves overall survival in lung (TC1), breast (4T1) and melanoma (B16) cancer models. To conduct this experiment, three days after tumor initiation, mice were intravenously injected with indicated glycolipids at 0.1 μgm glycolipid/mouse or vehicle once a week for 4 weeks.

### Example 3

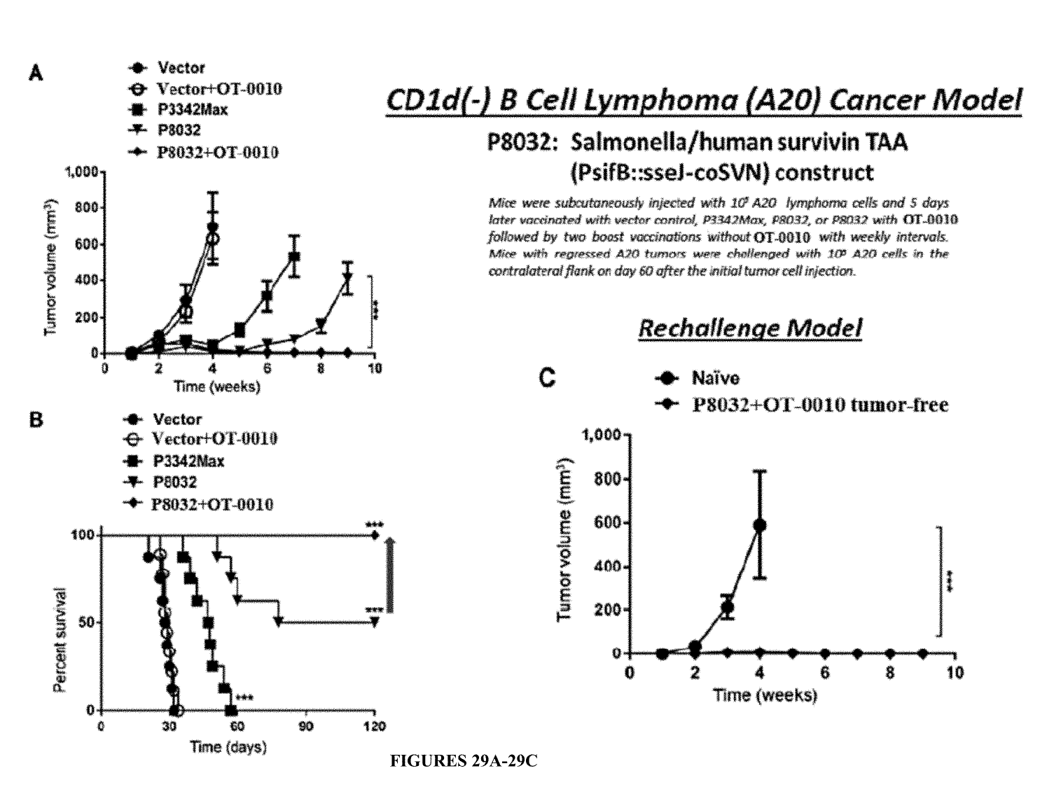

FIG. 29A-C shows that OT-0010 induces potent adaptive immune response and immune memory effect. The experiment was done using CD1d(−) B cell lymphoma (A20) cancer model. Mice were subcutaneously injected with 10⁵ A20 lymphoma cells and 5 days later vaccinated with vector control, P3342Max, P8032, or P8032 with OT-0010 followed by two boost vaccinations (without OT-0010) with weekly intervals. Mice with regressed A20 tumors were challenged with 10⁵ A20 cells in the contralateral flank on day 60 after the initial tumor cell injection.

### Example 4

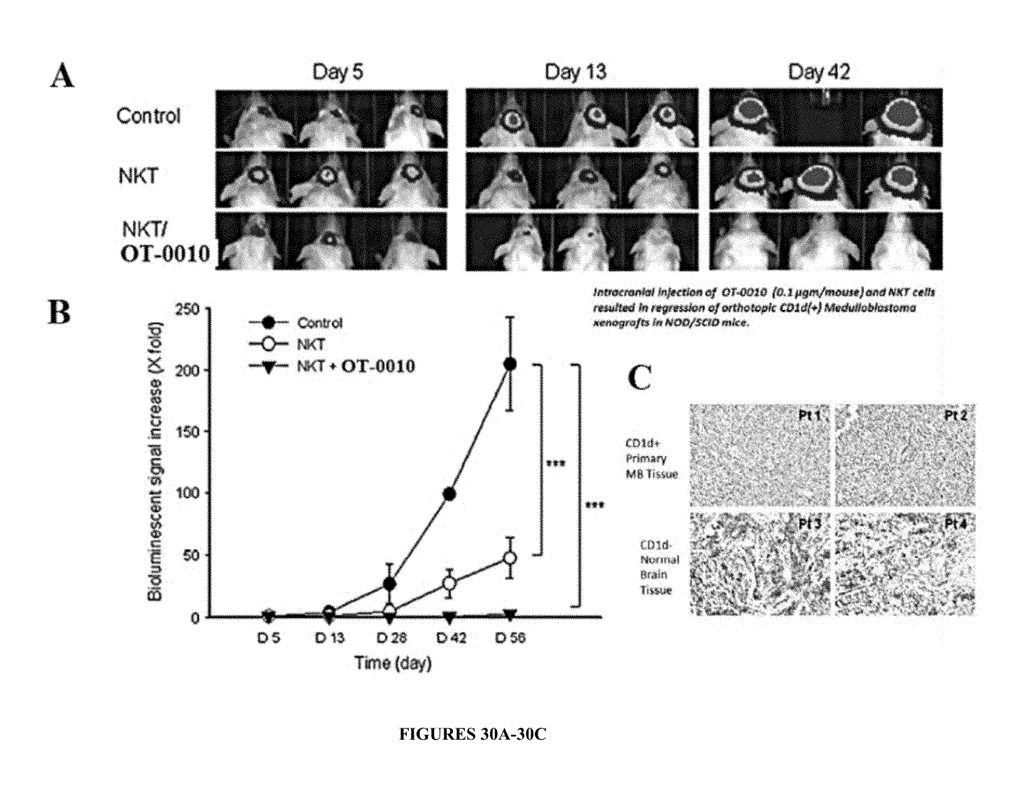

FIG. 30A-C shows that OT-0010 promotes potent NKT cell innate (direct) tumor cell killing. Intracranial injection of OT-0010 (0.1 gm/mouse) and NKT cells resulted in regression of orthotopic CD1d(+) Medulloblastoma xenografts in NOD/SCID mice.

### Example 5

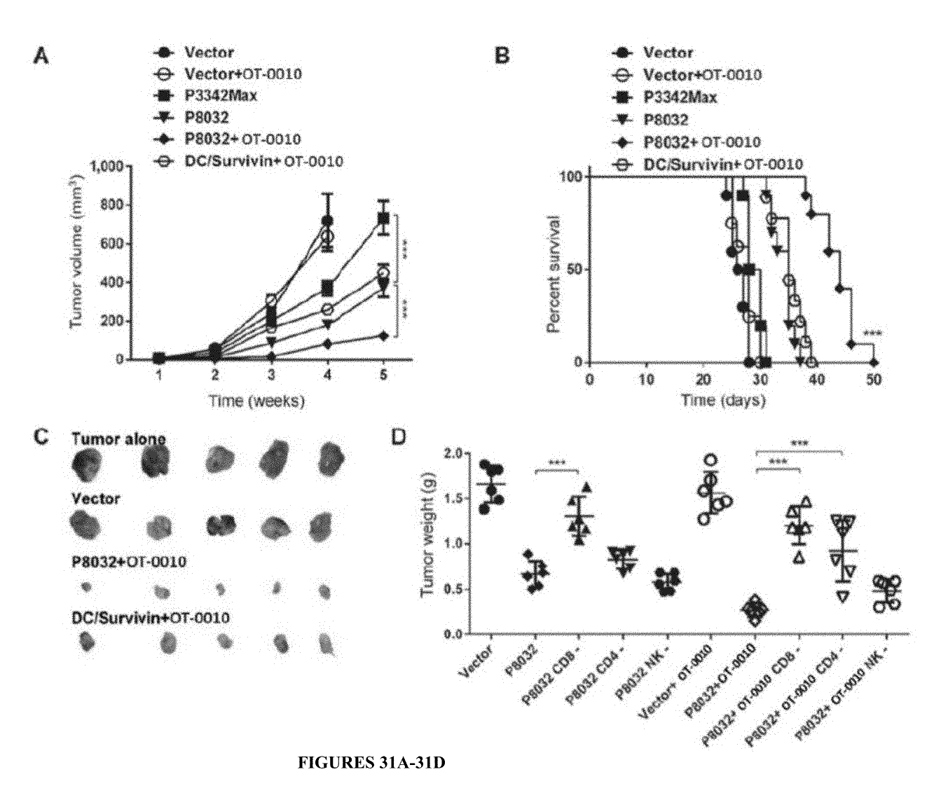

FIG. 31A-D shows that OT-0010 improves the overall survival in colon cancer (CT26) model. P8032 in this figure refers to Salmonella/human surviving TAA (PsifB::sseJ-coSVN) construct.

It is proposed that OT-0010 binds to antigen presenting dendritic cells in a CD1d-dependent manner which leads to dendritic cell maturation/activation and CD1d-restircted activation of NKT cells resulting in a potent Th-1 adaptive immune response via transactivation of NK cells and cytotoxic CD8+ T cells. Tumor cells express certain chemokines which serve as chemo-attractants to NKT cells promoting NKT cell migration into the tumor microenvironment (TME) and tumor stroma. Within the TME and tumor stroma, NKT cells encounter OT-0010 which is being presented by CD1d-positive (CD1d+) tumor cells and CD1d+ tumor associated macrophages (TAMs). NKT cells directly kill tumor cells and TAMs in a CD1d-restricted manner in the presence of OT-0010 resulting in a potent innate immune response.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A pharmaceutical composition comprising a therapeutically effective amount of a glycolipid of Formula I and a pharmaceutical carrier, wherein the therapeutically effective amount is the amount effective for shifting a Th2 immune response to a Th1 response by natural killer T cells (NKT cells) 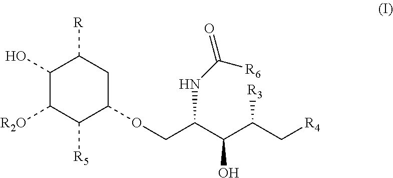
wherein, R═COOR₁ or CH₂OR₁;
R₁═H or an alkyl group;
R₂═H or SO₃⁻;
R₃═H or OH;
R₄═H, an alkyl group, an alkenyl group or an oxaalkyl group;
R₅═OH, acetamido or a halogen atom; and
R₆═X-A
A=
dialkyl phenyl; 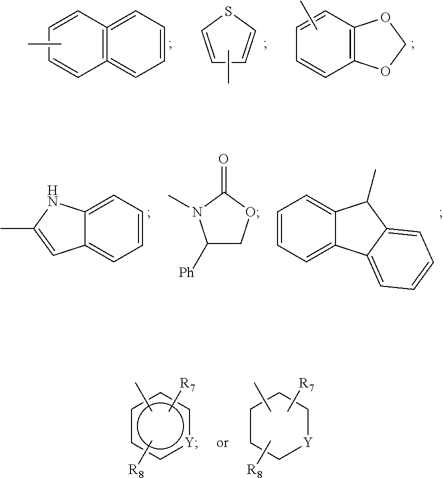
X=alkyl, alkenyl, alkoxy, thioalkoxy, substituted furan, or unsubstituted furan;
Y═N or C
R₇=halogen, H, phenyl, alkyl, alkoxy, nitro or CF₃; and
R₈=methyl or H;
or a pharmaceutically acceptable salt thereof.

2. A pharmaceutical composition comprising a therapeutically effective amount of a glycolipid of one or more of Formulas II-V or a pharmaceutically acceptable salt thereof, and a pharmaceutical carrier, wherein the therapeutically effective amount is an amount effective for shifting a Th2 immune response to a Th1 response by natural killer T cells (NKT cells)

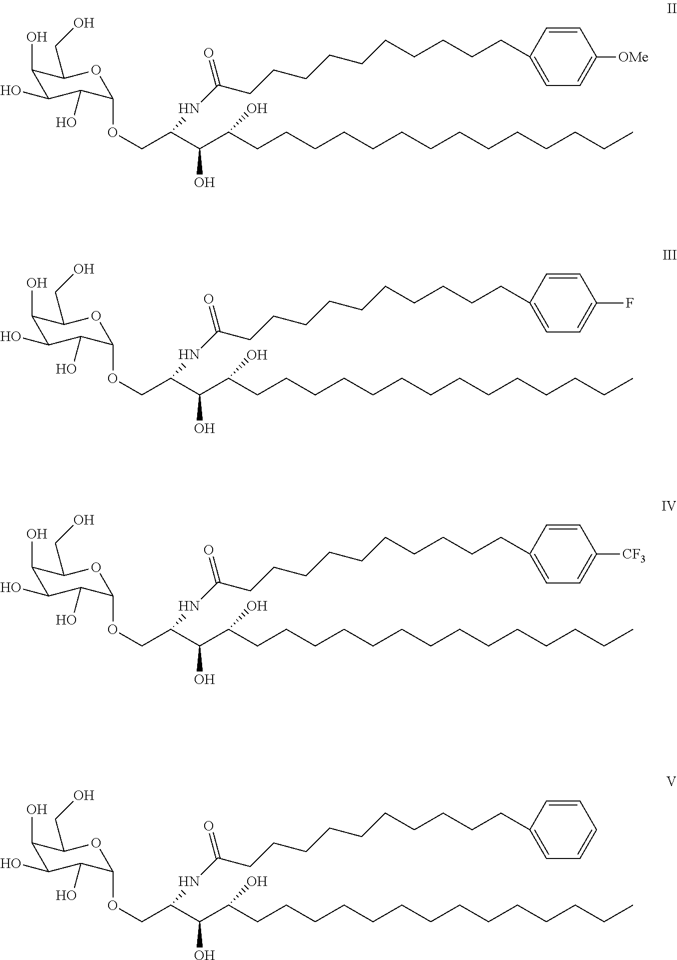 [SMILES: CCCCCCCCCCCCCC[C@@H](O)[C@@H](O)[C@H](COC1OC(CO)C(O)C(O)C1O)NC(=O)CCCCCCCCCCc1ccc(OC)cc1 ; CCCCCCCCCCCCCC[C@@H](O)[C@@H](O)[C@H](COC1OC(CO)C(O)C(O)C1O)NC(=O)CCCCCCCCCCc1ccc(F)cc1 ; CCCCCCCCCCCCCC[C@@H](O)[C@@H](O)[C@H](COC1OC(CO)C(O)C(O)C1O)NC(=O)CCCCCCCCCCc1ccc(C(F)(F)F)cc1 ; CCCCCCCCCCCCCC[C@@H](O)[C@@H](O)[C@H](COC1OC(CO)C(O)C(O)C1O)NC(=O)CCCCCCCCCCc1ccccc1]

3. A pharmaceutical composition comprising a therapeutically effective amount of a glycolipid of any one of Formulas I-V or a pharmaceutically acceptable salt thereof.

4. The pharmaceutical composition of any of claims 1-3, further comprising a therapeutic antibody, an antibody-drug conjugate, a radioimmunotherapy agent, a small molecule therapeutic agent or an immune stimulating agent.

5. The pharmaceutical composition of claim 4, wherein the pharmaceutical composition is administered to a subject having cancer.

6. The pharmaceutical composition of claim 5, wherein the cancer is a tumor, cancerous lesion, cancerous lymph node, or a circulating tumor cell (CTC).

7. The pharmaceutical composition of claim 5, wherein the cancer is selected from the group consisting of hematopoietic malignancies, prostate cancer, renal cancer, breast cancer, ovarian cancer, multiple myeloma, melanoma, colon cancer, lung cancer, cervical cancer, and certain neurological tumors, or metastases thereof.

8. The pharmaceutical composition of claim 4, wherein the therapeutic antibody further comprises a drug to form an antibody-drug conjugate.

9. The pharmaceutical composition of claim 4, wherein the pharmaceutical composition comprises a therapeutic antibody, and wherein the glycolipid is conjugated to the therapeutic antibody to form an antibody-glycolipid conjugate.

10. A method of treatment comprising administering the pharmaceutical composition of any of claim 1-4 or 9 to a subject in need thereof.

11. The method of claim 10, wherein the subject has cancer.

12. The method of claim 11, wherein the cancer is selected from the group consisting of hematopoietic malignancies, prostate cancer, renal cancer, breast cancer, ovarian cancer, multiple myeloma, melanoma, colon cancer, lung cancer, cervical cancer, and certain neurological tumors, or metastases thereof and metastases thereof.

13. The method of claim 10, wherein the glycolipid and therapeutic antibody are administered simultaneously or separately.

14. The method of claim 13, wherein the administration is by the same or by different routes of administration.

15. The method of claim 10, wherein the glycolipid and therapeutic antibody are combined in a single formulation.

16. The method of claim 10, wherein the glycolipid is administered prior to, simultaneously with or following administration of the therapeutic antibody.

17. A method for treating cancer by shifting a Th2 immune response to a Th1 response by natural killer T cells (NKT cells) comprising administering to a subject in need thereof, a therapeutically effective amount of a pharmaceutical composition of claims 1-3.
